(19) United States
(12) Patent Application Publication
(10) Pub. No.: US 2012/0202218 A1
(43) Pub. Date: Aug. 9, 2012

(54) DETECTION METHOD AND DEVICE BASED ON NANOPARTICLE AGGREGATION

(75) Inventors: Bo Liedberg, Linkoping (SE); Daniel Aili, Linkoping (SE); Lars Baltzer, Uppsala (SE); Karin Enander, Linkoping (SE); Johan Rydberg, Uppsala (SE)

(73) Assignee: MODPRO AB, Uppsala (SE)

(21) Appl. No.: 13/063,610

(22) PCT Filed: Sep. 14, 2009
(86) PCT No.: PCT/EP2009/061889

§ 371 Date: Jun. 1, 2011

(30) Foreign Application Priority Data
Sep. 12, 2008 (SE) 0801959-8

(51) Int. Cl. G01N 33/573; C40B 60/12; G01N 33/566; B82Y 15/00

(52) U.S. Cl. 435/7.4; 436/501; 506/39; 977/788; 977/920

(57) ABSTRACT

A method for determining the presence of a compound in a liquid solution, by admixing the liquid solution with a plurality of nanoparticles; providing conditions effective to cause aggregation of the nanoparticles in the liquid solution in the absence of said compound in the liquid solution; and observing a detectable signal reflecting the amount of aggregation of nanoparticles in the liquid solution, wherein the presence of the compound in the liquid solution results in a detectable signal reflecting a reduced amount of aggregation of nanoparticles in the liquid solution, in comparison to the amount of aggregation of nanoparticles obtained in the liquid solution in the absence of the compound therein. A nanoparticle, a composition, a kit and a for multi-well plate for use in the method are also disclosed. In some embodiments the association is cation-, anion and/or PH induced e.g. by using helix-loop-helix polypeptides as first and second molecules attached to the nanoparticles. The first molecules are directed to the target compound, the second molecules allow for aggregation of the nanoparticles.

### FIELD OF THE INVENTION

The present invention relates to the field of chemical and biochemical assays.

### BACKGROUND OF THE INVENTION

The interest for rapid, specific and inexpensive bioassays and sensors are steadily growing as new areas of applications, ranging from detection of narcotics and explosives to medical diagnosis and drug development, are identified. Conventional methods for protein analysis, such as ELISA,^{1,2} are often inexpensive and accurate but time consuming, whereas label free techniques based on surface plasmon resonance (SPR)³ and piezoelectric devices⁴ are generally rapid, but require, on the other hand, more complex fluidics and detection technologies. Recently, sophisticated and robust methods for protein sensing, similar to latex agglutination and sol particle immunoassays,^{5,6} but which exploit the colorimetric response of aggregating gold nanoparticles have been developed.⁷⁻¹⁰ The use of controllable aggregation of gold nanoparticles for sensor applications was pioneered by Mirkin et al,^{11,12} using DNA hybridization to induce an assembly of particles modified with single stranded DNA. Gold nanoparticles have an extraordinary high extinction coefficient, emanating from the plasmonic properties of the particles.¹³ Aggregation causes a large shift in the extinction spectrum that is manifested as a colour change of suspensions from red to purple, which facilitates very simple sensor readout.

This elegant technique has been utilized for the development of a large number of bioresponsive nanomaterials based on plasmonic nanoparticles modified with oligo-nucleotides, including peptide nucleic acid (PNA) and aptamers.¹⁴⁻¹⁶ Commonly, the particles are functionalized in such a way that the binding of a target compound induces aggregation. Nanoparticles are, however, very rarely stable as they might aggregate due to high salt concentrations or unspecific interactions, which then result in false positives. False positives may be more easily avoided in an assay where the particles are redispersed in the presence of the target compound,^{10,17} or aggregate in the absence of an analyte. Furthermore, in order to obtain a large colour change upon aggregation, the interparticle spacing must be small enough. The size of molecular recognition entities as “binders” frequently used for protein analysis in biosensors, e.g. antibodies and receptors, may prevent the particles from forming dense aggregates and the resulting colour change will hence be rather small. Suitable binders must therefore be relatively small in size, and of course be specific and facilitate simple immobilization without loosing their ability to recognize the target protein or decreasing particle stability.

Aili et. al describe how synthetic helix-loop-helix polypeptides may be utilized to control the assembly of gold nanoparticles and for biosensing³⁸. The authors describe gold nanoparticles having a plurality of a glutamic acid residue-rich polypeptide immobilized on the surface and show that the thus peptide-functionalized nanoparticles are very stable at neutral pH, but are caused to aggregate as the pH is reduced to about 4.6 or when introducing zinc ions into the solution.

### SUMMARY OF THE INVENTION

The present invention aims at providing nanoparticles having, attached to their surface, at least two different molecules, the first meolcules having an ability to cause the particles to aggregate in response to induction by a controllable parameter in a liquid phase, the second molecules having an ability to selectively bind a target present in the liquid phase. Such nanoparticles may be used in a wide variety of of chemical and biochemical assays, with a high sensitivity. The fact that the function of binding analyte and the function of causing the nanoparticles to aggregate are performed by different molecules provide for a wide versatility in the nanoparticle and represents a major expansion in the repertorire of analytes that can be measured using nanoparticles.

According to one aspect the invention provides a nanoparticle to which a plurality of molecules are attached, wherein the molecules attached to any one nanoparticle are capable of selectively binding a compound in a liquid solution and have an ability to associate with one or several molecules attached to any other of said nanoparticles. The attached molecules capable of selectively binding a compound in a liquid solution are not the same as the attached molecules having an ability to associate with one or several molecules attached to any other of said nanoparticles.

The attached molecules having an ability to associate with one or several molecules attached to any one other of said nanoparticles, comprise at least one moiety that enables cation-, anion- and/or pH-induced association between said molecules attached to different nanoparticles.

In one embodiment, the nanoparticle comprises attached molecules selected from proteins and/or polypeptides; e.g. the nanoparticle comprises attached molecules selected from helix-loop-helix polypeptides.

In one embodiment, the helix-loop-helix polypeptides are derived from a polypeptide according to any of the SEQ. ID. NOS. 4-31 by introducing an anchoring group for attachment of the polypeptide to the nanoparticle, wherein said anchoring group is either an amino acid residue of said sequence or a group attached to an amino acid residue of said sequence.

In one embodiment, the nanoparticle comprises molecules carrying a thiol, sulfide or disulfide function attached to the nanoparticle through a covalent bond.

In one embodiment, the nanoparticle comprises a plurality of attached polypeptides having an amino acid sequence containing a plurality of amino acids selected from glutamic acid and aspartic acid, such as to impart a net negative charge of from 4 to 10 to the polypeptide in a liquid solution at pH 7.

In one embodiment, the nanoparticle comprises a plurality of attached polypeptides derived from SEQ. ID. NO. 4, e.g. a plurality of attached polypeptides according to the SEQ. ID. NO. 2.

In one embodiment, the nanoparticle comprises a plurality of attached polypeptides that have an amino acid sequence derived from any one of SEQ. ID. NOS. 4-31 by introducing an anchoring group for attachment of the polypeptide to the nanoparticle, wherein said anchoring group is either an amino acid residue of said sequence or a group attached to an amino acid residue and wherein at least one amino acid residue of the polypeptide is functionalized by attaching a moiety capable of selectively binding a compound in a liquid solution.

In one embodiment, the nanoparticle comprises a plurality of attached functionalized polypeptides comprising a lysine, ornithine, diaminobutyric acid, or homolysine residue situated in position 34 that is funtionalized.

In one embodiment, the nanoparticle comprises a plurality of attached functionalized polypeptides comprising a histidine residue in a position i and a lysine, ornithine, diaminobutyric acid, or homolysine residue situated in position i+4 and/or in position i−3 that are functionalized.

In one embodiment, the nanoparticle comprises a plurality of attached functionalized polypeptides comprising a histidine residue in a position 11 and a lysine, ornithine, diaminobutyric acid, or homolysine residue situated in position 15 and/or in position 8 that are functionalized.

In one embodiment, the nanoparticle comprises a plurality of attached functionalized polypeptides comprising a moiety capable of selectively binding a compound dissolved in a liquid solution that is attached to an amino acid residue through a linking chain of formula —CₙH₂ₙ—, wherein n is 1-10.

In one embodiment, the nanoparticle comprises a plurality of attached functionalized polypeptides derived from a sequence according to any one of SEQ. ID. NOS. 23-28.

In one embodiment, the nanoparticle comprises a plurality of attached functionalized polypeptides derived from a sequence according to any one of SEQ. ID. NOS. 29-31, e.g. derived from SEQ. ID. NO. 30.

In one embodiment, the nanoparticle comprises a plurality of attached molecules capable of selectively binding an antibody.

In one embodiment, the nanoparticle comprises a plurality of attached molecules capable of selectively binding a compound selected from a protein, polypeptide, DNA, RNA, PNA, or carbohydrate.

According to another aspect, the invention provides a method for determining the presence of a compound in a liquid solution, by admixing the liquid solution with a plurality of nanoparticles according to the invention, providing conditions effective to cause aggregation of the nanoparticles in the liquid solution in the absence of said compound in the liquid solution; and observing a detectable signal reflecting the amount of aggregation of nanoparticles in the liquid solution,

wherein the presence of the compound in the liquid solution results in a detectable signal reflecting a reduced amount of aggregation of nanoparticles in the liquid solution, in comparison to the amount of aggregation of nanoparticles obtained in the liquid solution in the absence of the compound therein.

In one embodiment of the method, the conditions effective to cause aggregation of the nanoparticles in the liquid solution in the absence of said compound in the liquid solution are provided by adding a soluble salt to the liquid solution and/or by changing the pH of the liquid solution.

The soluble salt e.g. is a salt of a cation selected from Ca²⁺, Ni²⁺, Mg²⁺, Zn²⁺, La³⁺ and Fe³⁺, e.g. it is Zn²⁺.

In one embodiment, the conditions effective to cause aggregation in the absence of the compound in the liquid solution are provided by admixing the liquid solution with the plurality of nanoparticles.

The detectable signal e.g. may be the colour of the liquid solution, e.g. the change of the colour of the liquid solution, e.g. occurrence or non-occurrence of a colour change.

The detectable signal e.g. may be observed in the liquid solution.

The method may comprise depositing a drop of the solution on a solid surface so as to obtain a coloured spot on said solid surface, and observing the colour of the spot on the solid surface.

The method of the invention permits to determine the presence and optionally concentration of a compound such as a protein or polypeptide, a DNA, RNA, PNA, or a carbohydrate in a liquid solution.

According to a further aspect, a multi-well plate having a plurality of wells wherein each well holds a composition comprising a plurality of nanoparticles according to the invention is provided.

According to still a further aspect a kit comprising at least one container, the container holding a composition comprising a plurality of nanoparticles according to the invention is provided.

According to still a further aspect, a composition comprising a plurality of nanoparticles according to the invention in a liquid vehicle is provided.

Further aspects and embodiments of the invention will present themselves to the skilled reader of the following description and are as defined in the claims.

### BRIEF DESCRIPTION OF THE DRAWINGS

FIG. 1 Controlled aggregation of polypeptide functionalized gold nanoparticles enables a strategy for specific colorimetric protein sensing. The particles were modified with a polypeptide designed to allow a folding induced particle aggregation triggered by Zn²⁺, and a polypeptide based synthetic receptor for recognition of human carbonic anhydrase II (HCAII). (a) In the absence of HCAII, addition of Zn²⁺ induces a rapid colour shift from red to purple. (b) In the presence of HCAII, binding of the protein to the benzenesulphonamide ligand on the sensor scaffold obstructs the formation of dense aggregates which prevents the colour shift and the dispersions remain red.

FIG. 2 UV-vis spectra of particles with 10% KE2C-C6 at pH 7 (a) before ( - - - ) and after (—) addition of 70 nM HCAII. Inset: Closer view on the induced LSPR peak-shift caused by the binding of HCAII to the particles. (b) Spectra recorded with 2 min intervals for 20 minutes after diluting the particles in a buffer (pH 7) containing 10 mM Zn²⁺ without HCAII and (c) after incubating the particles in a sample containing HCAII. Concentration of HCAII was 70 nM after dilution.

FIG. 3 Shift in plasmon peak position a (λ_{Extmax}) as a function of time for particles functionalized with 10% KE2C-C6 and diluted in a Zn²⁺-containing buffer (bis-tris pH 7) in the absence of HCAII () and in the presence of 0.7 μM HCAII (□). Particles functionalized with 10% KE2C lacking the benzenesulphonamide ligand in the presence of 0.7 μM HCAII and 10 mM Zn²⁺ (Δ).

FIG. 4 Electron micrographs of the sensor peptide functionalized particles (a) and (b) in the presence of 3.4 μM HCAII and (c) without HCAII. The average interparticle distance (D) in the absence of HCAII was estimated to 2.4±0.1 nm corresponding to the size of a folded four-helix bundle of two JR2EC polypeptides,¹⁸ whereas in the presence of HCAII the interparticle distance was significantly larger as is evident in (d), showing the count as a function of D.

FIG. 5(a) The shift in plasmon peak position (λ_{Extmax}) as function of time for gold nanoparticles with 10% KE2C-C6 in the presence of 0.7 μM HCAII (□), 0.3 μM HSA () or 0.13 μM IgG (Δ). (b) The corresponding dot blots obtained by drying 3 μl of the suspensions on a nitrocellulose membrane. (c) SPR-sensorgrams reflecting the difference in association of 0.7 μM HCAII (), 0.3 μM HSA (—) and 0.13 μM IgG ( - - - ) to a gold surface with 10% KE2C-C6 (molar concentrations corresponds to 20 μg/ml).

FIG. 6 The influence of the HCAII concentration on the LSPR-maximum of particles with 10% KE2C-C6 after dilution in a Zn²⁺-containing buffer. Broken line drawn as guide for the eye.

FIG. 7(a) The influence of the Fab57P concentration on the LSPR-maximum of particles functionalized with JR2EC (∘) and JR2EC: C-pTMVP (20:1) () after being diluted in a Zn²⁺ containing buffer. (b) Corresponding normalized and smoothed extinction spectra of the JR2EC: C-pTMVP (20:1) functionalized particles in the presence of 100 nM (∘), 50 nM (), 25 nM (▪), and 0 nM (□) Fab57P.

FIG. 8(a) Position of LSPR peak maxima of gold nanoparticles functionalized with pTyr-PT (b) after being diluted in a buffer containing Mg²⁺ at various concentrations at pH 7. The particles aggregate at concentrations of Mg²⁺≧50 μM.

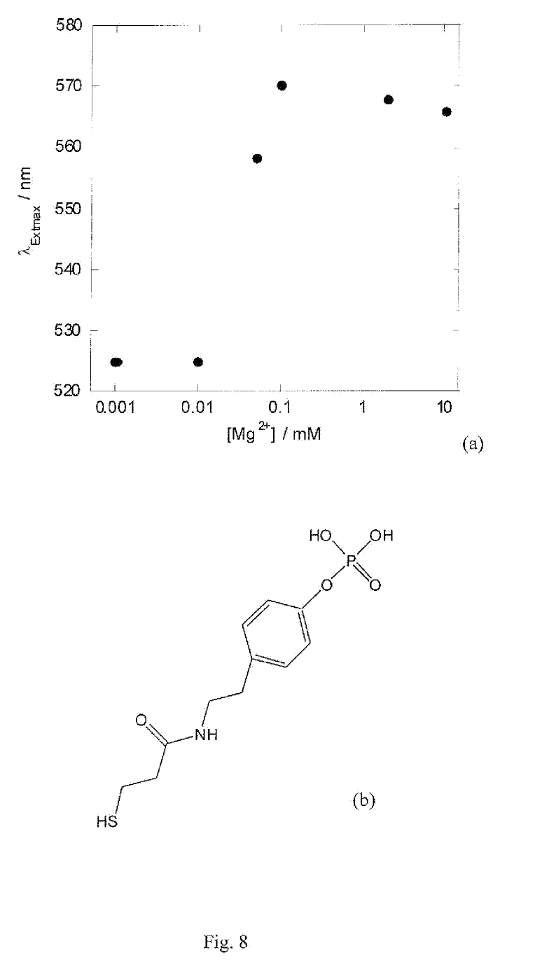

### DETAILED DESCRIPTION OF THE INVENTION

The Nanoparticle

According to a first aspect of the invention, a plurality of nanoparticles is provided, to which nanoparticles are attached

(i) first molecules that are capable of selectively binding a compound in a liquid solution; and

(ii) second molecules that have an ability to associate with one or several other second molecules attached to any other of the plurality of nanoparticles.

U.S. Pat. No. 6,361,944 to Mirkin et al., the contents of which is incorporated herein in its entirety, describes methods of preparing nanoparticles suitable for use in chemical detection methods, viz. for detecting oligonucleotides. In said US patent, the nanoparticles that are useful include nanoparticles made of e.g. metal (e.g., gold, silver, copper and platinum), semi-conductor (e.g., CdSe, CdS, and CdS or CdSe coated with ZnS) and magnetic (e.g., ferromagnetite) materials. Other nanoparticle materials useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃ As₂, InAs, and GaAs.

The nanoparticle according to the present invention is composed of gold, silver or gold and silver alloys or a combination of gold, silver or gold and silver alloys with a material selected from other metals, semiconductors, insulators, polymers and combinations thereof, e.g. materials as described in U.S. Pat. No. 6,361,944 to Mirkin et al. The nanoparticle may be a core/shell nanoparticle having either a shell or a core of gold, silver or gold and silver alloys. In one preferable embodiment, the nanoparticle is a core/shell particle having a gold surface. In another preferable embodiment, the nanoparticle is a gold nanoparticle. In yet another preferable embodiment, the nanoparticle is composed of a gold, silver or gold/silver alloy with a non-metallic shell, e.g. of a polymer or silica.

Core/shell nanoparticles and methods for producing the same also are described in e.g. U.S. Pat. No. 7,238,472 B2 to Mirkin et al., incorporated herein by reference.

Nanoparticles may furthermore be purchased from e.g., Ted Pella, Inc. (gold), Amersham Corporation (gold) and Nanoprobes, Inc. (gold).

The size of the nanoparticles according to the present invention suitably ranges from about 5 nm to about 150 nm (mean diameter in case of non-spherical nanoparticles), e.g. from about 5 to about 50 nm, in particular from about 10 to about 30 nm, or about 10 to 20 nm, especially 10 to 15 nm.

The nanoparticle of the invention has a plurality of molecules attached to the surface. Methods for preparing nanoparticles having molecules attached to the surface are known in the art. For instance, molecules functionalized with alkanethiols readily attach to gold or silver surfaces on nanoparticles. Other functional groups for attaching polypeptides to nanoparticles include e.g. sulfides and disulfides.

To prepare a nanoparticle to which a plurality of at least two different molecules are attached, the nanoparticle may be reacted with known amounts of these two molecules either in a common reaction step or in subsequent steps.

The nanoparticles coated with molecules should be sufficiently stable to allow storage without aggregation or with only reversible aggregation until use. For example, the nanoparticle of the invention suitably is stored in a liquid solution under conditions that do not allow for any substantial aggregation, e.g. a buffered aqueous solution. The nanoparticles also may be preserved as a lyophilized powder for reconstitution in a suitable solvent before use, e.g. in a buffered aqueous solution, such as an aqueous solution containing Bis-Tris buffer at pH 7.

The Molecules Attached to the Nanoparticles

For the purpose of the present invention, the expression “molecules attached to nanoparticles” and similar expressions do not refer to the target compound, albeit this latter will be able to bind to the recognition molecule, which in turn is attached to the nanoparticle. Instead, these expressions refer to molecules that are attached to the nanoparticles in a manufacturing process, such as the one described herein and by following the general methods known to the skilled person. The molecules attached to the nanoparticles preferably are attached by a covalent bond to the nanoparticle surface material.

At least a fraction of the molecules attached to the nanoparticles should have a specific binding affinity for the target compound and at least a fraction of the molecules attached to the nanoparticles should have an ability to associate with one or several molecules attached to any other of said nanoparticles under conditions effective to cause aggregation of the nanoparticles.

For the purpose of the present invention, the molecule having a specific binding affinity for the target compound is termed “recognition molecule”, while the molecule having an ability to associate with one or several molecules attached to any other of said nanoparticles is termed “aggregating molecule”.

For example, the molecules attached to the nanoparticle may comprise from about 1% to 99%, or about 10% to about 90%, or about 20 to about 80%, or about 30 to about 70% of recognition molecules, and from about 1% to 99%, or about 10% to about 90%, or about 20 to about 80%, or about 30 to about 70% of aggregating molecules, based on the total number of molecules attached to the nanoparticle.

At least two different types molecules are attached to the nanoparticles, at least one type having a specific binding affinity for the target compound (the recognition molecule), and at least one other type having an ability to associate with one or several molecules attached to any other of said nanoparticles (the aggregating molecule). Thus, it is a feature of the present invention that the recognition and aggregation function is effected by the different molecules, i.e. the recognition molecule is not identical to the aggregating molecule. In other words, at least two different types molecules are attached to the nanoparticles, at least one type having a specific binding affinity for the target compound (the recognition molecule), and at least one other type having an ability to associate with one or several molecules attached to any other of said nanoparticles (the aggregating molecule).

The total amount of molecules attached to the nanoparticle should be such as to provide the required function of controllable aggregation and recognition of target compound. The skilled person will easily verify that this is achieved by testing the nanoparticles using suitable reference solutions.

In one embodiment, the nanoparticle comprises at least one attached molecular species, having either recognition or aggregation function, which is a polypeptide or protein.

Any such polypeptide or protein, whether it is for the purpose of aggregation or recognition, may be attached to the nanoparticle by an anchoring group, naturally present in the an amino acid of the polypeptide, or introduced therein by derivatization, said anchoring group comprising a suitable functionality, as described e.g. in U.S. Pat. No. 6,361,944 to Mirkin et al. or in WO 03/080653.

One way of providing an anchoring group permitting to attach the polypeptide to the nanoparticle, is by introducing into the sequence of the polypeptide an amino acid with a high affinity for the nanoparticle surface, e.g. a Cys, Lys or Glu residue, or a non-natural amino acid comprising a corresponding functional group. The non-natural amino acid also may e.g. have an aminooxy function. Another way is to attach a bifunctional linker molecule to an amino acid residue in the polypeptide. Preferably, the anchoring group has been introduced site-specifically into the polypeptide scaffold by attachment of a bifunctional molecule to an amino acid residue capable of reacting chemoselectively or site-selectively with the bifunctional linker molecule according to the principles described above. The amino acid or linker molecule should be able to form a strong chemical bond to the nanoparticle surface onto which the polypeptide is to be attached, and this could be achieved by means of e.g. an SH, COOH, NH₂, biotin, his-tag, maleimide, fatty acid, or cholesterol moiety etc. The bifunctional linker molecule may have the general structure X—Rₙ—Y, wherein X is a functional group of the type COOH, NH₂, SH, SSAr (wherein Ar is an aromatic moiety, such as phenyl), CHO, CH₂Br, CH₂Cl, or CH₂I, Rₙ is an alkylene (CₙH₂ₙ) chain or ethylene glycol chain comprising n carbons, wherein n is a number ranging from 1 to 10, or 1 to 6, such as 1-4, and Y is a group of the type COOH, NH₂, SH, biotin, biotin analogue, His-tag, maleimide, silane, fatty acid or cholesterol.

Any polypeptide or protein may be attached to the nanoparticle by any of the functionalities as mentioned herein above, or as described e.g. in U.S. Pat. No. 6,361,944 to Mirkin et al and references therein.

For instance, molecules functionalized with alkanethiols readily attach to nanoparticles having at least a surface layer of gold and/or silver. Other functional groups for attaching polypeptides to nanoparticles include e.g. sulfides and disulfides.

In one embodiment, at least one of the proteins and/or polypeptides attached to the nanoparticle is a polypeptide having a helix-loop-helix fold or being capable of folding into a helix-loop-helix motif under suitable conditions, e.g. suitable pH, and being capable of dimerizing to form an antiparallel four-helix bundle, e.g. 37-47-amino acid polypeptide, or a 40-44-amino acid polypeptide, such as 42-amino acid polypeptide.

Such polypeptide may be derived e.g. from any of the polypeptides described in WO 07/117215 (Titled: Binder for C-reactive protein), WO 03/080653 (Titled: Novel polypeptide scaffolds and use thereof), WO 03/044042 (Titled: Site selective acylation), WO 01/085906 (Titled: Catalytically active peptides), WO 01/085756 (Titled: Site-selective acyl transfer), and WO 00/032623 (Catalytic peptides consisting of a designed helix-loop-helix motif and their use), (all incorporated herein by reference) using methods for functionalizing as described therein.

The derivatization may comprise introducing an anchoring group, as exemplified herein above, permitting to attach the polypeptide to the nanoparticle, suitably in the loop region, e.g. at an amino acid situated in a position, in a 42-amino acid helix-loop-helix polypeptide, of from position 18 to position 26, or from position 20 to position 24, e.g. in position 21, 22 or 23.

In one embodiment, the polypeptide is derived from any of the polypeptides according to SEQ. ID. NOS. 1-18 of WO 07/117215, or is derived from any of the polypeptides according to SEQ. ID. NOS. 1-4 of WO 03/080653, or is derived from any of the polypeptides according to SEQ. ID. NOS. 1-7 of WO 03/044042.

To prepare a nanoparticle having attached to it at least two different molecules, e.g. two different polypeptides, one being an aggregating molecule and one being a recognition molecule, the “naked” nanoparticle may be incubated in a solution, e.g. a suitably buffered aqueous solution, comprising known amounts of the molecules, for a suitable period of time, e.g. 2-24 hours, or 6-12 hours. The solution may comprise a 1:100 to 100:1 mixture of aggregating and recognition molecules, or a 1:20 to 20:1 mixture thereof, or a 1:10 to 10:1 mixture thereof, or a 1:5 to 5:1 mixture thereof or a 1:2 to 2:1 mixture thereof, or a 1:1 mixture thereof, based on the molar concentrations of the two types of molecules. Generally, the molecules to be attached to the nanoparticles are added in a large excess in comparison to the nanoparticles, and at the end of the incubation any unbound molecule is washed away.

The Aggregating Molecules

The molecules that have an ability to associate with one or several molecules attached to any other of said nanoparticles under conditions effective to cause aggregation of the nanoparticles (herein below referred to as “aggregating molecules”) may be selected from organic or inorganic molecules.

The associative properties of the aggregating molecules, i.e. their ability to associate with one or several molecules attached to any other of said nanoparticles under conditions effective to cause aggregation of the nanoparticles suitably depends upon a controllable parameter, such as the ionic strength, the presence of a given metal cation, the pH, of the liquid sample solution.

Thus, for example, the aggregating molecules may be of an ionic nature, i.e. cationic or anionic. Electrostatic repulsion between the charged aggregating molecules will then prevent the nanoparticles from aggregating. By adding a soluble salt containing an ion of opposite charge to a solution containing the charged nanoparticles, charge neutralization will occur and the nanoparticles will be able to aggregate.

For example, the aggregating molecules may be of the type having a moiety that is able to bind to the particle surface, such as a thiol-, sulfide- or disulfide-functional moiety, linked to at least one ionic moiety through a linker group, such as a C1-C10, e.g. a C1-C6, or C1-C4 aliphatic or aromatic moiety, e.g. an alkylene moiety (of the type —CₙH₂ₙ—).

In one embodiment, the aggregating molecules are C1-C10, or C1-C6, or C1-C4 alkylthiols, alkylsulfides or alkyldisulfides, substituted with at least one ionic function.

In one embodiment, the aggregating molecules are C1-C10, C1-C6, or C1-C4 alkylthiols; C1-C10, C1-C6, or C1-C4 alkylsulfides; or C1-C10, C1-C6, or C1-C4 alkyldisulfides substituted in w (omega) by an ionic function.

In one embodiment, the aggregating molecules are of anionic nature. Electrostatic repulsion between the negatively charged aggregating molecules will then prevent the nanoparticles from aggregating. By adding a soluble salt containing a cation to a solution containing the negatively charged nanoparticles, charge neutralization will occur, which will be inducive of aggregation of nanoparticles.

Examples of negatively charged functional groups that may be carried by the aggregating molecules are phosphate, sulphate, nitrate, carboxylate, phosphonate, and sulphonate groups.

When the nanoparticles carry negatively charged aggregating molecules, a soluble salt containing a cation selected from e.g. Na⁺, K⁺, Ca²⁺, Ni²⁺, Mg²⁺, Zn²⁺, Al³⁺, La³⁺, Fe³⁺; e.g. from Ca²⁺, Ni²⁺, Me²⁺, Zn²⁺, La³⁺ and Fe³⁺, suitably is added to the solution containing the nanoparticles, to induce aggregation.

In another embodiment, the aggregating molecules are of cationic nature. Electrostatic repulsion between the positively charged aggregating molecules will then prevent the nanoparticles from aggregating. By adding a soluble salt containing an anion to a solution containing the positively charged nanoparticles, charge neutralization will occur, which will be inducive of aggregation of nanoparticles.

Examples of positively charged functional groups that may be carried by the aggregating molecules are ammonium and guanidinium groups.

When the nanoparticles carry positively charged aggregating molecules, a soluble salt containing an anion selected from e.g. a halogen anion (e.g. fluoride or chloride), nitrate, phosphate and sulphate suitably is added to the solution containing the nanoparticles, to induce aggregation.

In one embodiment, the nanoparticles carry more than one type of aggregating molecules. For example, it is contemplated that the nanoparticles may carry an ionic molecule, such as described herein above, that prevents aggregation of the nanoparticles until conditions effective to cause aggregation are provided, in combination with a molecule that does not give rise to any such ionic repulsion, but which has an ability to associate with one or several molecules attached to any other of the nanoparticles. In this embodiment, there thus may be a separation of the two functions of the aggregating molecules, viz. that of preventing aggregation (until the assay is to be performed) and that of allowing the nanoparticles to aggregate in the absence of target compound (when performing the assay).

In one embodiment, the nanoparticle comprises an aggregating molecule that is a polypeptide or protein. The polypeptide or protein suitably has an ability to associate with one or several aggregating molecules attached to any other of said nanoparticles by forming a multimeric complex therewith under conditions that are permissive for such association, thereby inducing aggregation of the nanoparticles. For example, the polypeptide or protein attached to one nanoparticle may be one having an ability to dimerize with a polypeptide or protein attached to another nanoparticle under conditions permissive for such dimerization, i.e. under conditions effective to cause aggregation of the nanoparticles.

In one embodiment, the tertiary structure and/or net ionic charge of the polypeptide or protein attached to a nanoparticle is such as not to allow the polypeptide or protein to associate to any measurable degree with one or several molecules, e.g. proteins or polypeptides, attached to any other of said nanoparticles in the absence of conditions allowing aggregation of the nanoparticles. In this embodiment, bringing the liquid sample solution to conditions allowing aggregation of the nanoparticles causes a change in said tertiary structure and/or net ionic charge, thereby increasing the ability of the protein or polypeptide to associate with one or several molecules, attached to any other of said nanoparticles to such a degree that, in the absence of the target compound, the nanoparticles are caused to aggregate.

In one embodiment, at least one of the aggregating proteins and/or polypeptides attached to the nanoparticle is a 37-47-amino acid polypeptide, e.g. a 40-44 amino acids polypeptide, such as a 42 amino acids polypeptide, said amino acid polypeptide having a helix-loop-helix fold or being capable of folding into a helix-loop-helix motif under suitable conditions, e.g. suitable pH and/or presence of ionic substances.

Such aggregating polypeptide may be derived e.g. from any of the polypeptides described in the WO pamphlets referred to herein above, using methods for functionalizing as described therein, by providing, in addition to an anchoring group permitting to attach the polypeptide to the nanoparticle surface, functionalities that are able to prevent aggregation of the nanoparticles until conditions effective for aggregation of the nanoparticles are provided, e.g. acid functions such as provided by glutamic and/or aspartic acid, e.g. 3-15, or 4-12, e.g. 5-10, glutamic and/or aspartic amino acids.

In one embodiment, the design of the polypeptide sequences used to control nanoparticle aggregation is based on sequences previously shown to fold into helix-loop-helix motifs and dimerise to form four helix bundles²³. An important property of these polypeptides is that they fold as a consequence of dimerization while in their monomeric state they remain unordered. It was shown recently that homodimerization can be inhibited by the specific introduction of charged residues of the same sign at the interface between the two monomers³⁹. Negatively charged residues at the interface, i.e. in a and d positions of the heptad repeat commonly used to describe helical structure, will repel one another and prevent dimerization and thus folding, and positively charged residues will have the same effect. The sequence JR2E is an example of the former⁴¹. Full inhibition of dimerization is only achieved if all interface positions are filled with charged residues and the introduction of charged residues in other positions have little or no effect on dimerization. At a pH where the carboxylate groups become protonated there is little or no repulsion between the monomer subunits and the sequences will fold and dimerize as shown for JR2E. However, a reduction in pH is often detrimental to structure and affinity and the introduction of external agents, for example a Zn salt, to assist folding is advantageous as it eliminates the need to change pH in order to induce aggregation. The introduction of metal ion assisted folding of polypeptides to control nanoparticle aggregation is therefore an important feature of one particularly advantageous embodiment of the detection system according to the invention.

In one embodiment, the aggregating polypeptide is derived from any of the polypeptides according to SEQ. ID. NOS. 1-18 of WO 07/117215, from any of the polypeptides according to SEQ. ID. NOS. 1-4 of WO 03/08653, or from any of the polypeptides according to SEQ. ID. NOS. 1-7 of WO 03/044042.

In one embodiment, the aggregating polypeptide is derived from SA-42 (SEQ. ID. NO. 4), by introduction of an anchoring group, e.g. by replacing any of the amino acids in positions 18-26, or 20-24, e.g. 21-23, with an amino acid permitting the polypeptide to be attached to a nanoparticle and by introducing into the amino acid sequence a number of amino acids that permit to control the aggregation of the nanoparticles. These for example may be amino acids that give the peptide a net charge at selected conditions of e.g. pH, ionic strength or concentration of selected ions in the solution, so as to prevent homodimerization and, thereby, nanoparticle aggregation. In one embodiment, the amino acids that give the peptide a net charge are aspartic and/or glutamic acids.

In one particular embodiment, the aggregating molecule is a polypeptide according to SEQ. ID. NO. 2.

For the purpose of the present invention, and unless otherwise specified herein, the term “amino acid” refers to a natural, standard or non-standard amino acid, or a non-natural amino acid.

Examples of amino acid residues that may be present in the helix-loop-helix polypeptide to provide a controllable net negative electric charge are glutamic acid and aspartic acid. Examples of amino acid residues that may be present in the helix-loop-helix polypeptide to provide a controllable net positive electric charge are lysine, arginine, ornithine. For example, the polypeptide may comprise a plurality of glutamic acid residues that give the peptide a net negative charge of e.g. 9 to 2, or 8 to 3, e.g. 7 to 4, such as 5 or 6, at neutral pH and thereby prevent homodimerization at this pH. Dimerization and folding, will then occur at pH<6, or at pH 7 in the presence of a suitable cation, such as Zn²⁺. The folding is primarily driven by the formation of a hydrophobic core made up by the hydrophobic faces of the amphiphilic helices.

The Recognition Molecule

If the target compound is present in the liquid sample solution, it will selectively bind to those molecules attached to the nanoparticle that have a specific binding affinity for the target compound. This will interfere with the capability of association between molecules attached to separate nanoparticles, and thereby will lead to a reduced amount of aggregation of the nanoparticles under the conditions effective to cause aggregation thereof, compared to the amount of aggregation of the nanoparticles in the absence of the target compound in the liquid sample solution.

The expression “reduced amount of aggregation”, as used herein, not only refers to a situation where the number of particles that are aggregated, out of the total number of particles that are present in the liquid phase, is reduced. The reduced amount of aggregation also may refer to a situation where the distance between aggregating particles is increased, leading to a reduced density of the aggregates, and/or where aggregates formed are smaller. Thus, for the purpose of the present invention, a reference to a “reduced amount of aggregation”, should be understood as referring to a reduced number of aggregated particles and/or an increased distance between aggregating particles and/or formation of smaller aggregates.

The binding affinity of the molecules attached to the nanoparticle should be essentially selective for the target compound, compared to other compounds that may be present in the liquid sample solution. Furthermore, the binding affinity should be such that the presence of the target compound may be determined at very low concentrations of the target compound in the liquid sample solution.

In one embodiment, the recognition molecule comprises an epitope for an antibody, capable of selectively binding to the antibody. For example, the recognition molecule may comprise an oligopeptide or polypeptide derived from a virus or a bacterium or any other microorganism, having a capacity of selectively binding to an antibody.

In one embodiment, the recognition molecule is a polypeptide constituting a helix-loop-helix motif having a capacity of dimerizing to form a four-helix bundle, and carrying a moiety, that allows for specific binding of the target compound. The helix-loop-helix polypeptide suitably is a polypeptide that has been site-selectively functionalized with a suitable recognition moiety by use of a method as described e.g. in the above-mentioned WO 03/080653 and WO 03/044042. For example, the recognition molecule may be derived from a 42-amino acid polypeptide according to any of SEQ. ID. NOS. 1-7 as disclosed in WO 03/044042. The polypeptide should be able of attaching to the nanoparticle, and for this purpose may be derivatized as discussed herein above, e.g. by inclusion of a cysteine residue in the loop region, in particular at a position ranging from 18 to 26, or 20 to 24, in particular position 21, 22 or 23.

In one embodiment, the recognition molecule is derived from a polypeptide according to SEQ. ID. NO. 30, by replacing any of the amino acids in position 18-26, or 20-24, e.g. 21-23, with an amino acid permitting the polypeptide to be attached to a nanoparticle, e.g. a cysteine residue, said recognition molecule carrying a recognition moiety attached to the amino acid in position 34 (Lys34).

In one embodiment, the recognition polypeptide is KE2C (SEQ. ID. NO. 1), carrying a recognition moiety attached to the amino acid in position 34 (Lys34).

Preferably the recognition moiety is localized at the side chain of a lysine residue. In a 42-amino acid polypeptide as referred to herein above, such as a lysine residue e.g. may be situated at a position 34. Also, in a helix-loop-helix polypeptide comprising a histidine residue at a position i, and a lysine residue at position i+4 and/or i−3, the recognition moiety suitably may be localized at the side chain of either or both of these lysine residues. As an example, i may be 11, and either one or both of the amino acids at position 8 and 15 may be lysine residues.

Thus, in KE2 (SEQ. ID. NO. 30) the lysine residue to which the recognition moiety is attached is Lys34 because it is preferentially acylated due to its low pKa value and in KE3 (SEQ. ID. NO. 31) this lysine residue is Lys8 because it is close to the His residue in position 11 that ensures the site selectivity.

It should be noted that the lysine residues may be replaced by an ornithine, diaminobutyric acid, or homolysine residue, since these amino acid residues are equally capable of being functionalized by a reaction as described e.g. in WO 03/044042.

The choice of the recognition moiety depends on the target molecule. For assays aimed at detection of enzymes the recognition moiety is chosen from their known inhibitors. For detection of proteins other than enzymes, that have high affinity ligands, the recognition moiety to be attached to the polypeptide is chosen from the known ligands of the protein. For carbohydrate binding proteins the recognition moiety is a carbohydrate. For DNA and RNA the ligand is DNA, RNA or PNA. For target proteins for which there are no known ligands, the recognition moieties to be attached to the polypeptide are several compounds from a combinatorial library. One example of such a recognition moiety is benzenesulfonamide which is an inhibitor of carbonic anhydrase II. Any thrombin inhibitor can be used recognition moiety for the detection of thrombin and protease inhibitors can be used for the detection of proteases.

In one embodiment, the recognition moiety carried by a recognition molecule, e.g. a polypeptide, is capable of binding to a protein, i.e. the assay is aimed at detection and optionally quantification of a “target” protein.

In one embodiment, the recognition moiety is attached to the polypeptide through a linking chain, e.g. an alkylene chain of general formula —CₙH₂ₙ—, wherein n is 1-10, e.g. 2-8, such as 2-6.

Thus, the nanoparticle of the invention carries attached to its surface, molecules as described herein above.

For example, in some embodiments, the nanoparticle of the invention carries, attached to its surface, as an aggregation molecule, a polypeptide derived from SA-42 (SEQ. ID. NO. 4) by inclusion of a number of acidic residues in the amino acid sequence sufficient to provide the polypeptide with a net negative charge of e.g. 2 to 8 units; in an aqueous solution at a pH above the pI of the polypeptide, e.g. a pH above 6, e.g. pH 7, which has been additionally modified by inclusion of an anchoring group, such as a cysteine residue, in the loop region (positions 18-26, or 20-24, e.g. 21, 22, or 23), permitting to attach the polypeptide to the nanoparticle.

In some embodiments, the nanoparticle of the invention carries, attached to its surface, as a recognition molecule, a polypeptide derived from any one of the polypeptides according to SEQ. ID. NOS. 4-31, e.g. SEQ. ID. NOS. 23-31, by site-selective inclusion in the amino acid sequence, e.g. at a lysine, ornithine, homolysine, or diaminobutyric acid residue situated in position 34, preferably a lysine residue, of a moiety capable of selectively binding the target compound; which polypeptide has been additionally modified by inclusion of an anchoring group, such as a cysteine residue, in the loop region (positions 18-26, or 20-24, e.g. 21, 22, or 23), permitting to attach the polypeptide to the nanoparticle.

In some embodiments, the nanoparticle of the invention carries, attached to its surface, as a recognition molecule, a polypeptide comprising an epitope for an antibody, capable of selectively binding to the antibody, and provided with an anchoring group permitting to attach the polypeptide to the surface of the nanoparticle.

In one embodiment, the nanoparticle of the invention carries, attached to its surface, at least two types of polypeptides: one is a polypeptide derived from SA-42 by inclusion of a number of acidic residues in the amino acid sequence sufficient to provide the polypeptide with a net negative charge in an aqueous solution at a pH above the pI of the polypeptide, e.g. a pH above 6, e.g. pH 7; the other one being a polypeptide derived from any one of the polypeptides according to SEQ. ID. NOS. 4-31, e.g. SEQ. ID. NOS. 23-31, by site-selective inclusion in the amino acid sequence, e.g. at a lysine, ornithine, homolysine, or diaminobutyric acid residue situated in position 34, preferably a lysine residue, of a moiety capable of selectively binding the target compound; wherein both polypeptides have been additionally modified by inclusion of an anchoring group, such as a cysteine residue, in the loop region (positions 18-26, or 20-24, e.g. 21, 22, or 23), permitting to attach the polypeptide to the nanoparticle.

In one embodiment, the nanoparticle of the invention carries, attached to its surface, at least two types of polypeptides: one is a polypeptide derived from SA-42 by inclusion of a number of acidic residues in the amino acid sequence sufficient to provide the polypeptide with a net negative charge of e.g. 2 to 8 units in an aqueous solution at a pH above the pI of the polypeptide, e.g. a pH above 6, e.g. pH 7; the other one being a polypeptide derived from LA-42b (SEQ. ID. NO. 27), by site-selective inclusion in the amino acid sequence, e.g. at a lysine, ornithine, homolysine, or diaminobutyric acid residue, preferably a lysine, situated in position 34, of a moiety capable of selectively binding the target compound, wherein both polypeptides have been additionally modified by inclusion of an anchoring group, such as a cysteine residue, in the loop region (positions 18-26, or 20-24, e.g. 21, 22, or 23), permitting to attach the polypeptide to the nanoparticle.

In one embodiment, the nanoparticle of the invention carries, attached to its surface, at least two types of polypeptides: one is a polypeptide derived from SA-42 (SEQ. ID. NO. 4) by inclusion of a number of acidic residues in the amino acid sequence sufficient to provide the polypeptide with a net negative charge of e.g. 2 to 8 units in an aqueous solution at a pH above the pI of the polypeptide, e.g. a pH above 6, e.g. pH 7, and which has been additionally modified by inclusion of an anchoring group, such as a cysteine residue, in the loop region (positions 18-26, or 20-24, e.g. 21, 22, or 23), permitting to attach the polypeptide to the nanoparticle; and, the other polypeptide comprising an epitope for an antibody, capable of selectively binding to the antibody, and provided with an anchoring group permitting to attach the polypeptide to the nanoparticle.

It should be realized that the polypeptides of the present invention, whether for recognition or aggregation purpose, may differ from any of the polypeptides according to the Sequence Listing by one or several substitutions, preferably conservative substitutions as well as deletions or additions, that do not substantially interfere with their function. Amino acid substitutions are defined as conservative in nature when the substituted amino acid has similar structural and/or chemical properties. Examples of conservative substitutions are that of an alanine with a valine, or an asparagine with a glutamine. Thus, a polypeptide that is useful for providing a recognition or aggregation molecule according to the present invention may have an identity of e.g. 60%, 70%, 80%, 85%, 90%, 95%, 97%, 98%, 99% or 100% with any of the polypeptides according to the Sequence Listing.

The Liquid Sample Solution

In some embodiments, the liquid sample solution may be provided as a liquid sample, optionally diluted or concentrated, obtained from a liquid solution to be analysed for the presence of the target compound, i.e. susceptible of containing the compound the presence of which is to be determined (the target compound).

In some embodiments, the liquid solution susceptible of containing the target compound e.g. may prepared by bringing a solid, semi-solid or liquid sample in contact with a liquid phase, so as to dissolve the solid, semi-solid or liquid sample or part of the sample in the liquid phase.

The liquid phase preferably is an aqueous, e.g. water or an aqueous solution of buffers and/or salts, or an organic solvent that is miscible with water, such as e.g. an alcohol or a ketone, e.g. ethanol or acetone.

In still other embodiments, the liquid solution may be e.g. a supernatant or a culture medium. The solid, semisolid or liquid sample may be a biological sample, such as a biological sample obtained from an animal or plant, e.g. from a mammal. For example, the sample may be a serological sample or a fraction or extract from such sample.

The liquid solution also may be prepared by extraction of a solid, semi-solid or liquid sample in a suitable extraction medium, e.g. a liquid solvent medium.

The liquid sample solution suitably is provided in an aqueous buffer medium, e.g. at pH 7.

The assay method according to the invention is highly suitable for determination of protein concentrations in a liquid solution. It can also be used for the determination of DNA, RNA and PNA concentrations, as well as concentrations of carbohydrates, e.g. oligo- and polysaccharides.

It lies well within the capability of the person of ordinary skill in the art to prepare and test solutions of known concentration of the target compound in order to verify the detection limit thereof with regard to the selected parameters of the assay system. It also lies well within the capability of the person of ordinary skill in the art to verify that false negatives or positives are not obtained when assaying a test sample solution.

For example, a reference solution may be based on nanoparticles lacking the recognition molecules. The reference assay to confirm that a positive response is not false suitably is peformed in parallel with the test assay, under identical conditions, but using nanoparticles lacking recognition molecules. In the absence of recognition molecules at the surface of the nanoparticles, target compound present in the sample solution will not be able to prevent aggregation of the nanoparticles and a positive answer will inevitably be due to a non-specific interaction.

The Assay Method

When performing the assay to the determine the presence of the target compound in the liquid sample solution, the liquid sample solution, containing the nanoparticles and the test sample, is observed for a detectable signal at conditions effective to cause aggregation of the nanoparticles in the absence of the target compound in the liquid sample solution.

In one embodiment, such conditions may be obtained e.g. by adjusting the pH or the ionic strength of the sample solution or the concentration of a metal ion therein, to within a range where the molecules attached to the nanoparticles become able to associate with molecules attached to separate nanoparticles.

The nanoparticles that are to be admixed with the liquid sample solution may be provided in a liquid vehicle, e.g. an aqueous solution containing a suitable buffer, such as a pH 7 buffer, e.g. pH7 Bis-Tris buffer. The liquid vehicle may be the same as the liquid vehicle of the liquid sample solution.

In one embodiment of the inventive method, the nanoparticles in a vehicle are added to the liquid sample solution. In another embodiment, the nanoparticles are provided in a liquid vehicle to which a volume of the liquid sample is added.

In one embodiment, after admixing the nanoparticles in a liquid vehicle and the test sample, the test sample solution is incubated for a sufficient period of time, to allow at least some of the target compound, if present in the test sample, to bind to the recognition molecules. The test sample solution, comprising the nanoparticles and the test sample, then is brought to conditions effective to cause aggregation of the nanoparticles in the absence of the target compound, and a detectable signal is observed, reflecting the amount of aggregation of nanoparticles in the liquid sample solution, i.e. indicative of the presence or absence of the target compound in the test sample, and optionally of the concentration of the target compound. The suitable incubation time may range from less than one minute to 30 minutes, e.g. from 1 minute to 20 minutes, or from 2 minutes to 10 minutes, and may be easily determined by using a reference solution comprising the target compound.

In another embodiment, the conditions allowing for aggregation of the nanoparticles are present already when admixing test sample and nanoparticles. In this embodiment, too, presence of target compound will lead to a reduced amount of aggregation in the test sample solution. The observed detectable signal may be compared with that obtained using a reference sample containing no target compound and/or a reference sample containing a known amount of target compound.

In one embodiment, the detectable signal is directly observable by the naked eye. For example, the detectable signal may be a change of optical properties of the liquid sample solution, e.g. a colour change. A positive result, i.e. indicating presence of the target compound in the liquid sample solution, may be observed as an absence of a colour change, or a reduced colour change, compared to the colour change obtained in the absence of the target compound.

In one embodiment of the method of the invention, nanoparticles carrying aggregating molecules of opposite net charge are used, which nanoparticles will tend to aggregate because of electrostatic attraction between oppositely charged nanoparticle.

In this embodiment, the liquid solution to be assayed for the presence of a target compound suitably is first admixed with a plurality of nanoparticles carrying a net charge of one sign, and subsequently a plurality of nanoparticles carrying a net charge of the opposite sign are added.

The detectable signal also may be observed using a suitable instrument, such as an instrument permitting to register an optical signal, e.g. a spectrophotometer. For example, the presence or absence of the target compound may be determined as a function of the observed wavelength shift of the extinction maximum measured.

The presence of the target compound in the test sample solution may be observed by the colour of the solution remaining red, while the absence of the target compound in the test sample solution may be observed as a colour change, from red to purple. The colour or wavelength shift signals obtained using nanoparticles having a surface of gold, silver or of a gold and silver alloy are essentially similar.

In some cases, e.g. when the target compound is of a somewhat smaller size, a colour change may be observed also in the presence of the target compound, albeit slighter than in the absence of the target compound.

In all cases, by comparing the detectable signal observed using different reference solutions containing known amounts of target compound, the presence and/or amount of target compound in the sample solution may be determined.

The signal also may be one detectable by means of e.g. an optical instrument or any other instrument permitting to measure a parameter of the liquid sample solution linked to the amount of aggregation of the nanoparticles in the liquid sample solution.

The method of the invention may comprise a step of transferring solution onto a solid support, such as a nitrocellulose membrane, so-called dot.blotting, before observing the detectable signal on the solid support.

The detectable signal also may be observed directly in the reaction vessel, e.g. in a well of a multi-well plate.

In a high-throughput method, the detectable signal may be observed directly in the well of a multi-well plate using a suitable plate reader, such as the Safire² plate reader, commercially available from Tecan Trading AG, Switzerland.

In one embodiment of the invention, the concentration of the target compound in the test sample solution is determined. This may be done by measuring the detectable signal using reference solutions of known concentration of the target compound, e.g. 2-6 different concentrations, over the measurement range. At the same time, the detection limit of the specific assay may be determined.

By the method of the invention, various compounds may be detected and optionally quantified. For example, the inventive method allows to detect and optionally quantify various proteins and polypeptides, such as enzymes, antibodies, hormones, cytokines etc; nucleic acids, such as oligonucleotides and polynucleotides, e.g. DNA, RNA and, PNA etc, and carbohydrates, such as oligosaccharides and polysaccharides.

The Kit

According to one aspect of the invention a kit is provided, comprising at least one container, the container holding a composition comprising a plurality of nanoparticles having molecules attached thereto, the molecules attached to any one nanoparticle having a binding affinity for a target compound, the presence of which is to be determined in a test sample solution (i.e. the target compound), and having an ability to associate with one or several molecules attached to any other of said nanoparticles.

Thus, the kit of the invention comprises at least one container, the container holding a composition comprising a plurality of nanoparticles according to the invention, as described herein above.

In one embodiment, the kit comprises at least one container holding a composition comprising an agent that when admixed with the composition comprising the nanoparticles is capable of inducing aggregation of said nanoparticles.

In one embodiment, the agent is a soluble metal salt, such as a watersoluble salt of a cation selected from e.g. Na⁺, K⁺, Ca²⁺, Ni²⁺, Me²⁺, Zn²⁺, Al³⁺, La³⁺, Fe³⁺; e.g. from Ca²⁺, Ni²⁺, Mg²⁺, Zn²⁺, La³⁺ and Fe³⁺, e.g. a watersoluble zinc salt, such as e.g. ZnCl₂.

In another embodiment, the agent is an acid or an acid buffer permitting to reduce the pH of the test solution to a pH below 7, e.g. pH 6 or lower.

In one embodiment, the kit comprises a well or container for mixing the composition comprising a plurality of nanoparticles with a composition comprising the target compound, and the composition comprising an aggregation-inducing agent.

In one embodiment, the kit comprises a solid support, e.g. a nitrocellulose membrane, onto which a sample of the liquid solution may be spotted before observing a detectable signal, such as the colour of the spot obtained on the support.

The Multi-Well Plate

According to one aspect of the invention, a multi-well plate is provided, having a plurality of wells wherein each well holds a composition comprising a plurality of nanoparticles having molecules attached thereto, the molecules attached to any one nanoparticle having a binding affinity for a target compound, the presence of which is to be determined in a test sample solution (i.e. the target compound), and having an ability to associate with one or several molecules attached to any other of said nanoparticles.

In one embodiment of the multi-well plate, the nanoparticles are present in suitably buffered aqueous solution, such as an aqueous solution buffered at pH 7, e.g. containing a Bis-Tris pH 7 buffer.

The number of wells in the multi-well plate may range up to e.g. 1000 or more.

The multi-well plate may additionally comprise a number of reference wells holding a composition comprising a plurality of nanoparticles having molecules attached thereto, said molecules having an ability to associate with one or several molecules attached to any other of said nanoparticles, but wherein the nanoparticles do not have any recognition molecule attached to the surface, i.e. do not have any attached molecules that have a binding affinity for the target compound.

The multi-well plate of the invention suitably may be used in a high-throughput method and preferably is a microwell plate, having wells of e.g. a volume as low as 0.5 nanolitres, e.g. 0.5 to 5 nanolitres, e.g. 1-3 nanolitres, and up to 5-500 microlitres, e.g. 10-100 microlitres.

The invention will be better understood by the following examples, which are not to be construed as limiting for the invention, but as purely illustrative thereof. Thus, in Example 1, recognition of the target protein is carried out using a polypeptide modified with a benzenesulphonamide ligand and designed to selectively bind the enzyme human carbonic anhydrase II (HCAII). The interaction between HCAII and benzenesulphonamide is well-characterized and was selected for a proof of concept demonstration.¹⁹ This particular polypeptide previously has been utilized in a solution assay for HCAII based on fluorescence detection using environmentally sensitive probes for reporting binding events.²⁰⁻²² The advantage with the present approach is the simplicity of the readout which does not require any advanced equipment as it can be performed by the naked eye. In addition, gold nanoparticles are in contrast to fluorophores not susceptible to bleaching and can be used in complex environments such as serum. The binders are also small enough to allow formation of dense aggregates in the absence of the target compound resulting in large colorimetric shifts. Furthermore, the synthetic polypeptides that may be used in the method according to the present invention are easy and cheap to manufacture at a large scale and their robustness ensures a long shelf-life.

In Example 2, the versatility of the proposed strategy is further demonstrated using a second model system based on the recognition of a peptide sequence from the tobacco mosaic virus coat protein (TMVP) by a recombinant Fab fragment (Fab57P). The proposed strategy for biodetection based on the controlled aggregation of synthetic receptor functionalized gold nanoparticles is hence a generic platform that can be further developed for detection and quantification of a wide array of target proteins.

### EXAMPLE 1

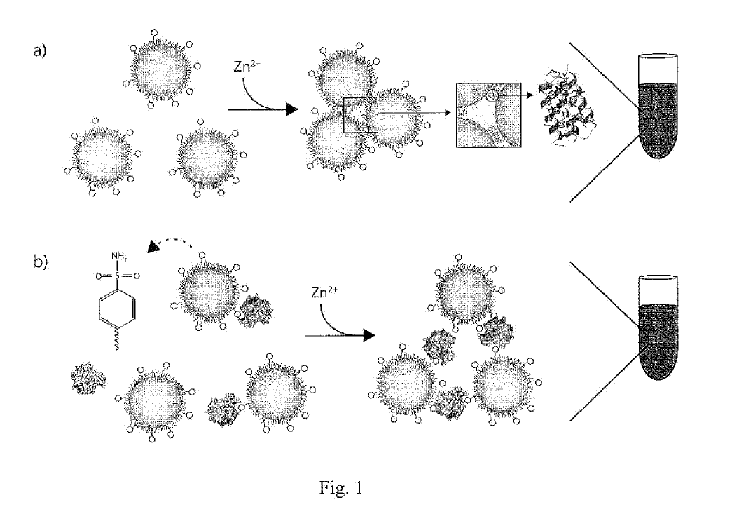

Polypeptide design: The design of JR2EC and KE2C was based upon the SA-42 and LA-42b polypeptide scaffolds, respectively. SA-42 is a 42 amino acid helix-loop-helix polypeptide that dimerizes in solution and folds into an antiparallel four-helix bundle.^{23,24} LA42b is a daughter sequence of SA-42 but modified in order to catalyze site-selective self-functionalization. JR2EC has a large number of Glu residues that give the peptide a net charge of −5 at neutral pH and prevent homodimerization.²⁵ Dimerization and folding, however, occur at pH<6 or at pH 7 in the presence of Zn²⁺.^{18,26} The folding is primarily driven by the formation of a hydrophobic core made up by the hydrophobic faces of the amphiphilic helices. The KE2C polypeptide was designed as a scaffold for biosensor applications and exists as a folded homodimer in solution at neutral pH.²²′²⁷ In order to allow specific binding of the target protein, KE2C was site selectively modified with a benzensulphonamide derivate with a six carbon aliphatic spacer (FIG. 1). The ligand was attached to the side chain of Lys34 using an orthogonal protection group strategy. Benzenulphonamide (H₂NO₂SC₆H₅) is a commonly used inhibitor for the 29 kDa, enzyme Human Carbonic Anhydrase II (HCAII) and has a reported K_{D} of 1.5 μM.²⁸ A considerably higher binding strength is obtained for benzenulphonamides that are para-substituted with an alkyl-residue, due to the additional binding energy provided by interactions with the hydrophobic CA binding cleft. The benzenulphonamide-derivatized scaffold, referred to as KE2C-C6, binds HCAII with a K_{D} of 0.02 μM,²¹ which is about one order of magnitude lower than the corresponding non-conjugated ligand.²⁹

Design and Performance of the Colorimetric Assay: The JR2EC, KE2C and KE2C-C6 polypeptides have a Cys residue in the loop region (position 22) which facilitates site specific and directed immobilization onto gold substrates. By co-immobilizing the sensor scaffold with JR2EC, particles with excellent stability, controllable aggregation properties and with a capability to selectively bind HCAII were obtained. In order to estimate the influence of ligand density on the particle response, the concentration ratio of KE2C-C6 to JR2EC in the loading solutions was varied between 0-100%. Although, the size and net charge of the two peptides are rather similar, the KE2-peptides are most likely folded whereas JR2EC mainly is random coil when immobilized at pH 6. The ratio of peptides in the loading solution may therefore not necessarily agree with the final surface concentration. The specified relative peptide concentrations thus correspond to the ratio of peptides in the loading solutions. In order to remove excess peptides from the solution, the particles were repeatedly centrifugated and resuspended in 30 mM bis-tris pH 7. The peptide decorated particles showed no traces of aggregation in bis-tris buffer whereas unmodified particles aggregated irreversibly upon transfer to the same buffer indicating that peptides were successfully immobilized. The calculated pI of JR2EC is 4.56,³⁰ and the peptide functionalized particles thus have a relatively high negative net charge and display good stability at neutral pH. The stability of the nanoparticles can be drastically reduced by protonating the acidic residues or by coordinating Zn²⁺. At neutral pH, Zn²⁺ not only trigger folding of JR2EC in solution but also when immobilized on gold nanoparticles.²⁶ The presence of Zn²⁺ results in a rapid but reversible nanoparticle aggregation induced by the dimerization and folding between peptide monomers immobilized on adjacent particles.

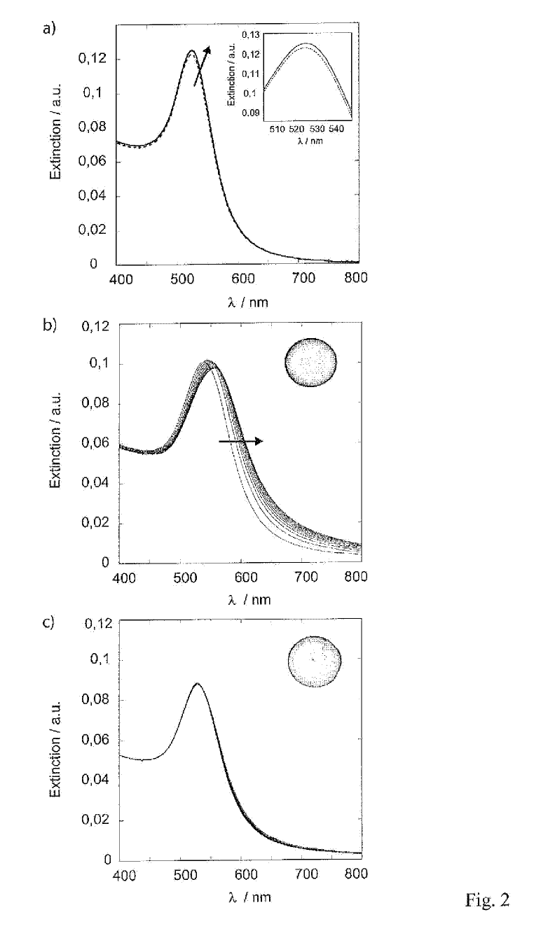

The ˜13 nm gold nanoparticles have a very pronounced extinction maximum close to 520 nm due to coherent electron oscillations referred to as localized surface plasmon resonance (LSPR). The position of the LSPR peak position a (λ_{Extmax}) and intensity is sensitive to changes in refractive index in the close vicinity of the particle surface.³¹ These shifts are generally very small and addition of 70 nM HCAII to a suspension of gold nanoparticles with 10% KE2C-C6 resulted in a barely visible redshift (˜1 nm) and a slight increase in intensity of the LSPR peak (FIG. 2a). No shift in peak position or increase in peak intensity was observed for particles without the benzenulphonamide ligand (data not shown).

In order to increase the magnitude of the LSPR-peak shift upon binding of HCAII, the influence of the protein on the extent of particle aggregation was examined. Aggregation causes a dramatic redshift of the LSPR-peak and the magnitude of the obtained shift is highly dependent on the interparticle distance and the size of the aggregates. The smaller the distance and the bigger the aggregates, the larger the resulting redshift.^{13,32,33} The steric constraints caused by the association of macromolecules to the surface of the particles results in a larger interparticle separation upon aggregation, which significantly decreases the extent of the LSPR-peak shift.

Particles decorated with both JR2EC and KE2C-C6 were very stable at pH 7 but aggregated immediately when subjected to Zn²⁺. The UV-vis spectra were rapidly redshifted to approximately 560 nm and the resulting colour shift from red to purple was clearly visible by the naked eye (FIG. 2b). A significantly smaller redshift (λ_{Extmax} ˜527 nm) was observed when first incubating the particles with a sample containing HCAII before diluting them in the Zn²⁺ containing buffer (FIG. 2c). The final concentration of HCAII after dilution was 70 nM. The distance between particles separated by the folded four-helix bundle is about 2.3±0.1 nm.¹⁸ As HCAII is approximately 47×41 Å in size,³⁴ the interparticle distance increases to a minimum of 5 nm when the protein is bound to the immobilized sensor scaffold. Taking into consideration the thickness of the polypeptide film, an additional ˜2-3 nm must be added to the interparticle separation. The binding of HCAII thus resulted in a significantly smaller redshift and the suspensions kept a distinct red colour (FIG. 2c, Inset).

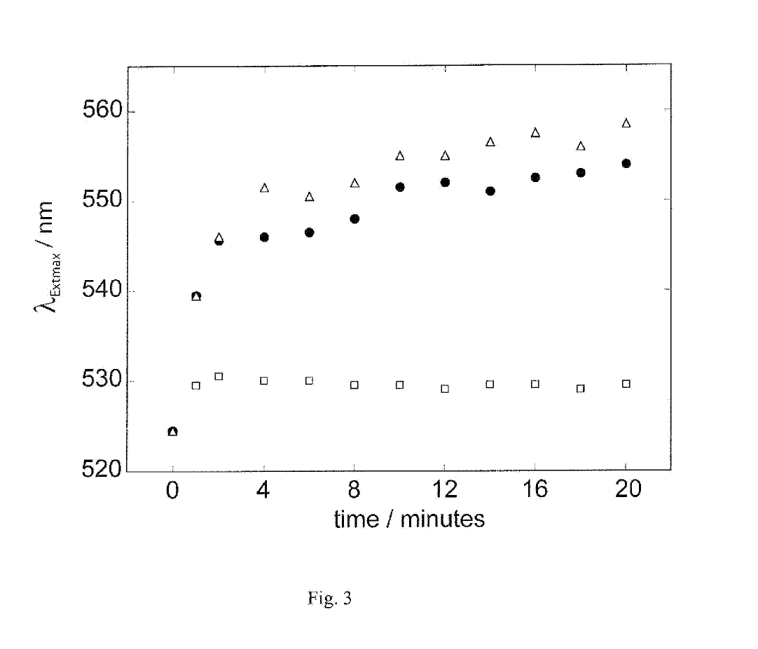

The redshift of the LSPR peak for particles functionalized with ratios of KE2C-C6 ranging from 0-100%, 20 minutes after dilution in a pH 7 buffer containing 10 mM Zn²⁺ in the absence and presence of HCAII is summarized in Table 1. No significant differences in stability between particles having different ratio of JR2EC to KE2C-C6 was observed in the absence of Zn²⁺. The extent and rate of aggregation in the presence of Zn²⁺, as well as the response to HCAII was similar for compositions of KE2C-C6 ranging from 10-50%. The particles remained dispersed during the time course of the measurements (20 minutes) indicating that the dissociation rate of the bound proteins was very slow (FIG. 3). At lower ratios of KE2C-C6 (<10%), the particles aggregated when subjected to Zn²⁺ due to the lack of binding sites for HCAII. Particles decorated only with the sensor peptide (100% KE2C-C6) also aggregated in the Zn²⁺-containing buffer but to a significantly lower extent and with a slower rate.

TABLE 1
λ_{Extmax} (nm) for particles functionalized
with varying amounts of KE2C-C6.

| % KE2C-C6 | pH 7 (nm) | +Zn²⁺ (nm) | +70 nM HCAII (nm) |
|---|---|---|---|
| 100 | 524.5 | 542 | 532 |
| 50 | 525 | 559 | 526 |
| 20 | 524 | 559 | 526 |
| 10 | 524.5 | 558.5 | 527 |
| 3.3 | 525 | 555 | 551 |
| 0 | 524 | 557.5 | 556 |

For particles functionalized with the KE2C polypeptide (10%) lacking the benzenulphonamide ligand but in the presence of 0.7 μM HCAII, the rate of aggregation was comparable to particles with KC2C-C6 (10%) in the absence of HCAII, indicating that the extent of unspecific association to the particles was very low (FIG. 3).

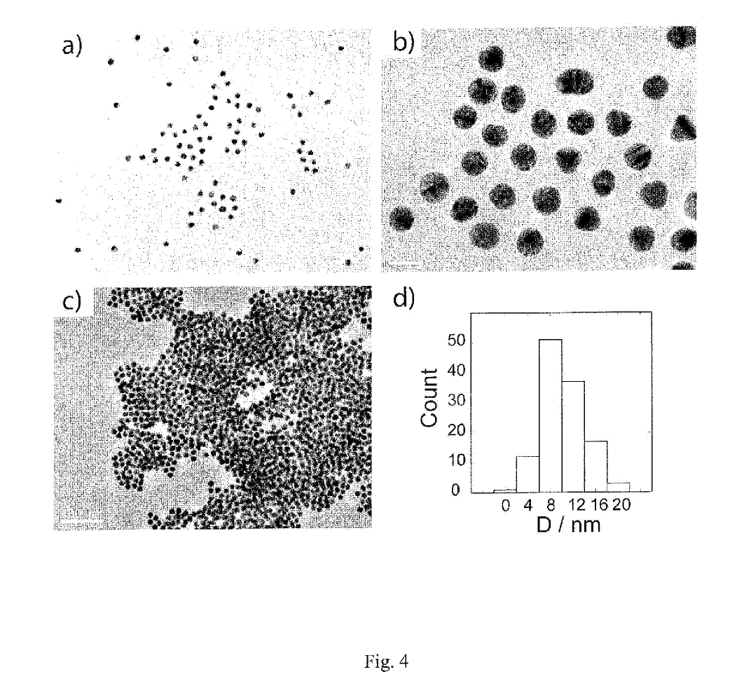

The separation of the aggregated particles with and without HCAII was estimated from electron micrographs (FIG. 4). In the absence of HCAII, large aggregates with an average interparticle distance of 2.4±0.1 nm, was obtained. This is in excellent agreement to what has previously been reported for aggregated particles separated by a folded four-helix bundle.¹⁸ After incubation in the presence of HCAII the aggregates were smaller and displayed a significantly larger interparticle separation. A large proportion of the particles were also not aggregated, presumably as the dimerization between JR2EC monomers in between the particles is prevented by the bound proteins (a sort of steric stabilization). The aggregates that did form are most likely a result of both unspecific effects (e.g. drying, bridging, and electrostatic interactions) as well as specific interactions (folding). The interparticle distance within the aggregates were distributed around 8-12 nm (FIG. 4d) which is reasonable for particles separated by 1-2 HCAII molecules.

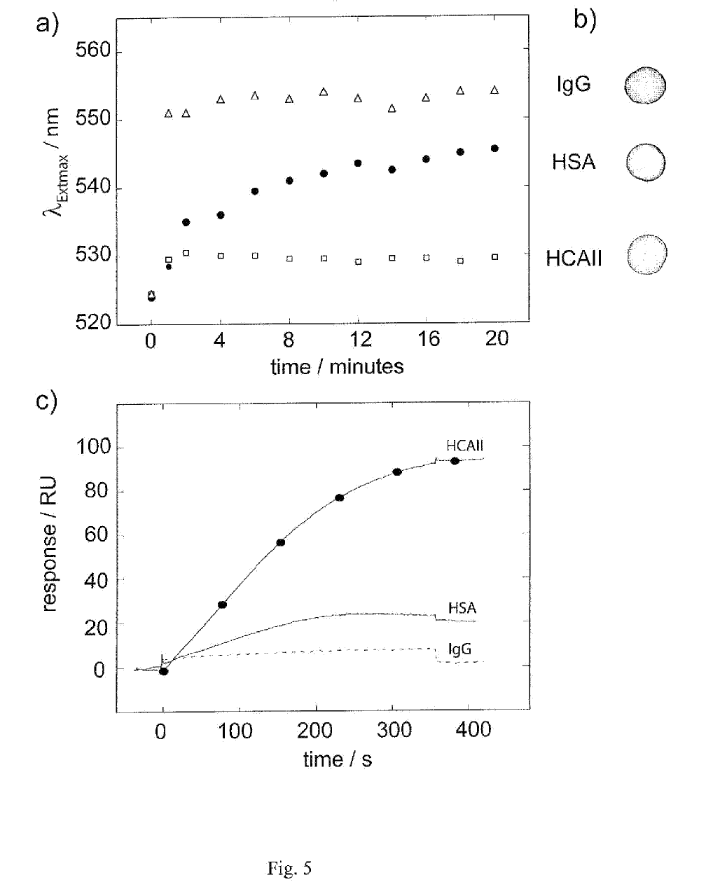

Although the extent of unspecific adsorption of HCAII to the particles was very low, the performance of a sensor in a complex environment is often highly influenced by the presence of other proteins. Human serum albumin (HSA) and Immunoglobulin G (IgG) are very abundant in e.g. serum. HSA bound to the particles but dissociated when the particles were diluted in the Zn²⁺-containing buffer, resulting in a gradually increasing redshift of the LSPR peak (FIG. 5a). The final LSPR shift at equilibrium was about 20% lower than in a protein-free sample. In the presence of IgG the particles demonstrated a slight increase in the initial aggregation rate, which might indicate some unspecific interaction between the IgG and the particles. The differences in colour of the dot blots were significant when comparing the three proteins IgG, HSA and HCAII where both IgG and HSA showed purple blots whereas the specifically bound HCAII gave rise to a red blot (FIG. 5b). The presence of both IgG and HSA (20 μg/ml) in the buffer seemed no to interfere with the binding of HCAII as no significant impact on the particle aggregation was observed.

The KE2 scaffold has previously been utilized for biosensor applications in solution and when immobilized in a solution-like hydrogel. The ability of the sensor scaffold to bind HCAII and to discriminate between different proteins when immobilized directly on bare gold surfaces was confirmed using surface plasmon resonance (SPR) (FIG. 5c). The polypeptides (JR2EC and KE2C-C6) were immobilized at a 10:1 ratio on thoroughly cleaned gold surfaces (Biacore, GE-Health Care) and the interactions with HCAII, HSA, and IgG were investigated using a Biacore 3000 instrument (Biacore, GE-Health Care). HCAII (0.7 μM) bound readily to the surface giving rise to a 100 RU response after a 5 minute injection. IgG did not show any specific interaction whereas HSA showed a slight association to the surface, about 15% of the response of HCAII, in agreement with the results obtained using the gold nanoparticles based assay.

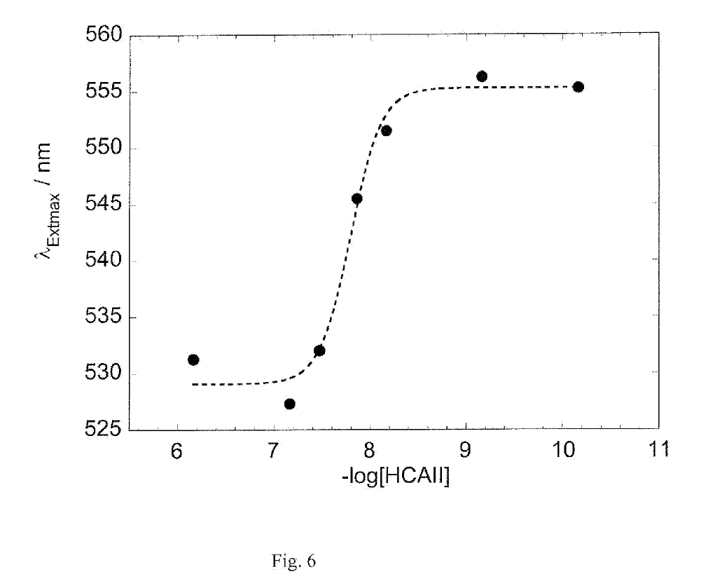

The extent of aggregation was dependent on the concentration of HCAII. The dynamic range stretches over approximately one order of magnitude (FIG. 6) which might be considered as rather narrow and the use of the present assay for quantification of the target protein is therefore limited. Strategies for altering the dynamic range may involve engineering of the ligand spacer length, which has previously been demonstrated to generate sensor peptides with affinities for HCAII ranging from 0.02-3μM.²¹ By co immobilizing sensor peptides having different affinities for the target compound the dynamic range of the assay can most probably be widened significantly.

### EXAMPLE 2

Antibody fragment assay: The versatility of the proposed sensing strategy is demonstrated using a second model system based on the recognition of a small peptide sequence from the tobacco mosaic virus coat protein (TMVP) by the recombinant antibody fragment Fab57P³⁵ derived from the monoclonal antibody Mab57P³⁶. The peptide C-pTMVP corresponds to amino acid 134-151 of TMVP and has been shown to bind Fab57P with a K_{D} in the 1 nM range.³⁵ The C-pTMVP peptide was modified with a Cys residue at the N-terminus and was co-immobilized with JR2EC on gold nanoparticles at a 1:20 ratio from a buffered pH 8.5 loading solution. At higher ratios of C-pTMVP the particles were not stable.

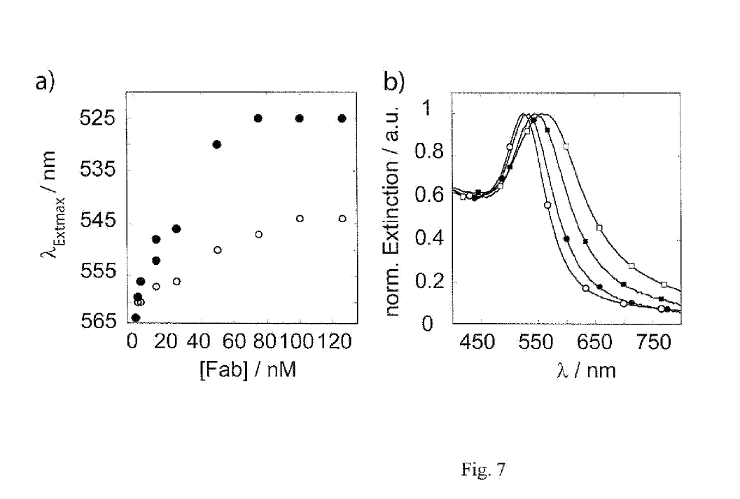

Although the unspecific adsorption of the Fab fragment to particles functionalized with only the JR2EC polypeptide was not negligible, the binding of Fab57P to the pTVMP-wt modified particles was significantly larger (FIG. 7). At a concentration of Fab57P of 100 nM the difference in the wavelength shift upon aggregation was ˜20 nm for particles with 0% and 5% C-pTMVP, respectively. The binding could thus readily be distinguished by the naked eye. Concentrations of Fab57P down to ˜25 nM could be detected. Measurements were performed on a 384 well plate format, ensuring very low sample consumption.

Conclusions: Aggregation of the particles was induced by a folding mediated dimerization between helix-loop-helix polypeptides immobilized on separate particles. Peptide-based synthetic receptors were utilized for the recognition of the target proteins human carbonic anhydrase II (HCAII) and the antibody fragment Fab57P. Binding obstructed the formation of dense particle aggregates which prevented the redshift observed in the absence of the proteins. The difference in colour could readily be seen by the naked and concentrations down to ˜15 nM of HCAII and ˜25 nM of Fab57P could be measured. The polypeptide modified particles are extremely stable under a wide variety of conditions and can be stored for several months without aggregating. The robustness of the synthetic receptors further ensures a long shelf-life. This strategy is simple and robust and can be tailored for detection of a wide variety of different proteins or other relevant bio-macromolecules using low cost equipment for detection.

Material and Methods

Proteins and peptides: HCAII and HSA were obtained from Sigma, and IgG from Octapharma. Fab57P was expressed and affinity purified as described.³⁷ The polypeptides The polypeptides KE2C (SEQ. ID. NO. 1: CH3CO-NAADLEAAIRHLAEKLAARGPCDAAQLAEQLAKKFEAFARAG-COOH), JR2EC (SEQ. ID. NO. 2: H2N-NAADLEKAIEALEKHLEAKGPCDAAQLEKQLEQAFEAFERAG-COOH), and C-pTMVP (SEQ. ID. NO. 3: H2N-CRGTGSYNRSSFESSSGLV-CONH2) were synthesized on a Pioneer automated peptide synthesizer (Applied Biosystems) using standard fluorenylmethoxycarbonyl (Fmoc) chemistry with O-(7-benzotriazole-1-yl)-1,1,3,3-tetramethyluronium tetrafluoroborate (TBTU, Alexis Biochemicals) as the activating reagent. The synthesis was performed on a 0.1 mmol scale with an Fmoc-Gly-PEG-PS (KE2C, JR2EC) or Fmoc-PAL-PEG-PS (C-pTMVP) resin (Applied Biosystems) and a fourfold excess of amino acid was used in each coupling. In KE2C, the final N terminus was capped with 0.3 M acetic anhydride in N,N-dimethylformamide (DMF). To allow site-selective incorporation of a benzenesulphonamide derivative in position 34 of KE2C, the side chain of Lys34 in this peptide was protected by an allyloxycarbonyl (Applied Biosystems). Otherwise, all amino acids were from Calbiochem-Novabiochem AG. Before cleaving KE2C from the resin, Lys34 was orthogonally deprotected for 3 h at room temperature using three equiv of tetrakis(triphenylphosphine)palladium(0) in a mixture of trichloromethane, acetic acid, and N-methylmorpholine (17:2:1 v/v; 12 mL per gram of polymer). The resin was washed sequentially with 20 mM diethyldithiocarbamic acid in DMF, 30 mM diisopropylethylamine (DIPEA) in DMF, DMF and dichloromethane (DCM), and desiccated. A fraction of the resin was then mixed with two equivalents of a pre-activated benzenesulphonamide derivative (H2NO2SC6H4CONH(CH2)5CONHS)21 and five equivalents of DIPEA in DMF (6 mL per gram of polymer) and was left to stand with occasional swirling for 12 h at room temperature to yield KE2C-C6. The resin was washed in DCM and desiccated.

KE2C, KE2C-C6, JR2EC and C-pTMVP were cleaved from the resin by treatment with a mixture of trifluoroacetic acid (TFA), ethanedithiol, water, and triisopropylsilane (94:2.5:2.5:1 v/v; 15 mL per gram of polymer) for 2 h at room temperature. After filtration, TFA was evaporated and the peptides were precipitated by the addition of cold diethyl ether, centrifuged, resuspended in diethyl ether and lyophilized. The crude products were purified by reversed-phase HPLC on a semi-preparative C-8 column. Peptides were eluted with a 40-minute gradient of 30-50% aqueous 2-propanol and 0.1% TFA (KE2C, KE2C-C6 and JR2EC) or isocratically at 18% aqueous acetonitrile and 0.1% TFA (C-pTMVP). Purified peptides were identified by MALDI-TOF mass spectrometry. The concentration of the peptides when dissolved was estimated under the assumption that they contained 25% water in the lyophilized state.

Particles Synthesis and Functionalization: Gold nanoparticles with an average diameter of ˜13 nm were prepared by citrate reduction of HAuCl₄ as previously described.²⁶ The UV-vis spectra of the prepared particles showed a distinct extinction maximum at 518-520 nm. Functionalization with JR2EC, KE2C and KE2C-C6 was performed by incubating the particles in a peptide solution (total concentration: 100 μM) at pH 6.0 (10 mM citrate) over night. C-pTMVP was co-immobilized with JR2EC at 1:20 concentration ratio (total peptide concentration: 100 μM) from a buffered pH 8.5 solution (10 mM borate). To remove unbound peptide remaining in solution, the particles were repeatedly centrifugated (˜18000 g) and the supernatant was removed and replaced with fresh 30 mM bis-tris buffer pH 7.0 until the resulting concentration of peptides in solution was less than 0.5 nM.

Characterization techniques: UV-vis spectroscopy was performed on a Schimadzu UV-1601PC spectrophotometer with 0.5 nm resolution at room temperature. The particle concentration was ˜0.5 nM when conducting the UV-vis experiments. TEM was conducted on a Philips CM20 Ultra-Twin lens high-resolution microscope operating at 200 kV. Each sample (20 μl) was incubated on carbon coated TEM-grids for 2 minutes before the suspension was removed using a filter paper and the grids were dried. For SPR analysis, the polypeptides were immobilized by incubating thoroughly cleaned gold substrates (Biacore, GE Health Care, Uppsala, Sweden) in a buffered pH 7 loading solution containing 100 μM JR2EC and KE2C-C6 at a 10:1 molar ratio for 16 hours at room temperature. The surfaces were rinsed and sonicated in ultra pure water (MilliQ, 18 MΩ cm) before being mounted on a chip holder. SPR sensorgrams were recorded using a Biacore 3000 instrument (Biacore, GE Health Care, Uppsala, Sweden) operating at a wavelength of 760 nm and equipped with four flow channels. HBS-N (10 mM Hepes, 0.15 M NaCl) was used as running buffer and the flow rate was 20 μl/minute. Dot-blotting was performed by first incubating the polypeptide functionalized particles for 2 minutes with the samples, before being diluted 1:50 in a pH 7 buffer containing 10 mM Zn²⁺. The diluted samples were then incubated for at least 5 minutes before putting 3 μl of the suspensions onto a nitrocellulose membrane (Amersham Biosciences) which was allowed to dry before being scanned. Analysis of the TVMP functionalized particles were performed in a 384 well plate using a Safire² plate reader (Tecan Trading AG, Switzerland).

In the above example, decorating the gold nanoparticles with two or more sequences, one of them controlled by external agents such as Zn ions for their folding and dimerization, very advantageously eliminates the dependence on pH for nanoparticle aggregation. While a change in pH can be a useful strategy for control of aggregation of the nanoparticles it may also have a negative effect on the stability of the proteins to be analyzed by causing them to denature or change conformation, thereby causing release of the proteins from the sequences introduced for recognition and binding or irreversible denaturation that will make the test inoperable. The use of a separate control peptide for nanoparticle aggregation therefore represents a major expansion in the repertorire of analytes that can be measured using this principle.

### REFERENCES

(1) Engvall, E.; Perlmann, P. Immunochemistry 1971, 8, 871-874.
(2) Van Weemen, B. K.; Schuurs, A. H. W. M. FEBS Lett. 1971, 15, 232-236.
(3) Liedberg, B.; Nylander, C.; Lundström, I. Sens. Actuators 1983, 4, 299-304.
(4) Ebersole, R. C.; Ward, M. D. J. Am. Chem. Soc. 1988, 110, 8623-8628.
(5) Singer, J. M.; Plotz, C. M. Am. J. Med. 1956, 21, 888-892.
(6) Leuvering, J. H. W.; Thal, P. J. H. M.; van der Waart, M.; Schuurs, A. H. W. M. J. Immunoassay Immunochem. 1980, 1, 77-91.
(7) Hirsch, L. R.; Jackson, J. B.; Lee, A.; Halas, N. J.; West, J. L. Anal. Chem. 2003, 75, 2377-2381.
(8) Thanh, N. T. K.; Rosenzweig, Z. Anal. Chem. 2002, 74, 1624-1628.
(9) Tsai, C.-S.; Yu, T.-B.; Chen, C.-T. Chemical Communications 2005, 4273-4275.
(10) Laromaine, A.; Koh, L. L.; Murugesan, M.; Ulijn, R. V.; Stevens, M. M. Journal of the American Chemical Society 2007, 129, 4156-4157.
(11) Mirkin, C. A.; Letsinger, R. L.; Mucic, R. C.; Storhoff, J. J. Nature 1996, 382, 607-609.
(12) Storhoff, J. J.; Elghanian, R.; Mucic, R. C.; Mirkin, C. A.; Letsinger, R. L. Journal of the American Chemical Society 1998, 120, 1959-1964.
(13) Storhoff, J. J.; Lazarides, A. A.; Mucic, R. C.; Mirkin, C. A.; Letsinger, R. L.; Schatz, G. C. J. Am. Chem. Soc. 2000, 122, 4640-4650.
(14) Stewart, M. E.; Anderton, C. R.; Thompson, L. B.; Maria, J.; Gray, S. K.; Rogers, J. A.; Nuzzo, R. G. Chem. Rev. 2008, 108, 494-521.
(15) Rosi, N. L.; Mirkin, C. A. Chemical Reviews 2005, 105, 1547-1562.
(16) Eugenii Katz, I. W. Angew. Chem., Int. Ed. 2004, 43, 6042-6108.
(17) Liu, J.; Lu, Y. Angew. Chem., Int. Ed. 2006, 45, 90-94.
(18) Aili, D.; Enander, K.; Rydberg, J.; Lundström, I.; Baltzer, L.; Liedberg, B. J. Am. Chem. Soc. 2006, 128, 2194-2195.
(19) A. Elisabeth Eriksson, P. M. K., T. Alwyn Jones, Anders Liljas, Proteins 1988, 4, 283-293.
(20) Enander, K.; Dolphin, G. T.; Andersson, L. K.; Liedberg, B.; Lundström, I.; Baltzer, L. J. Org. Chem. 2002, 67, 3120-3123.
(21) Enander, K.; Dolphin, G. T.; Liedberg, B.; Lundström, I.; Baltzer, L. Chem-Eur J 2004, 10, 2375-2385.
(22) Enander, K.; Dolphin, G. T.; Baltzer, L. J. Am. Chem. Soc. 2004, 126, 4464-4465.
(23) Olofsson, S.; Johansson, G.; Baltzer, L. J. Chem. Soc. Perk. T. 2 1995, 2047-2056.
(24) Olofsson, S.; Baltzer, L. Fold. Des. 1996, 1, 347-356.
(25) Nilsson, K. P. R.; Rydberg, J.; Baltzer, L.; Inganas, O. Proc. Natl. Acad. Sci. U.S.A. 2003, 100, 10170-10174.
(26) Aili, D.; Enander, K.; Rydberg, J.; Nesterenko, I.; Björefors, F.; Baltzer, L.; Liedberg, B. J. Am. Chem. Soc. 2008, 130, 5780-5788.
(27) Enander, K.; Dolphin, G. T.; Andersson, L. K.; Liedberg, B.; Lundström, I.; Baltzer, L. Journal of Organic Chemistry 2002, 67, 3120-3123.
(28) Taylor, P. W.; King, R. W.; Burgen, A. S. V. Biochemistry 1970, 9, 2638-2645.
(29) King, R. W.; Burgen, A. S. V. Proc. R. Soc. Lond. B 1976, 193, 107-125.
(30) Gasteiger, E.; Hoogland, C.; Gattiker, A.; Duvaud, S.; Wilkins, M. R.; Appel, R. D.; Bairoch, A., In The Proteomics Protocols Handbook, 2005 ed.; Walker, J. M., Humana Press, 571-607.
(31) Willets, K. A.; Van Duyne, R. P. Annu. Rev. Phys. Chem. 2007, 58, 267-297.
(32) Su, K. H.; Wei, Q. H.; Zhang, X.; Mock, J. J.; Smith, D. R.; Schultz, S. Nano Lett. 2003, 3, 1087-1090.
(33) Rechberger, W.; Hohenau, A.; Leitner, A.; Krenn, J. R.; Lamprecht, B.; Aussenegg, F. R. Opt. Commun. 2003, 220, 137-141.
(34) A. Elisabeth Eriksson, T. A. J., Anders Liljas, Proteins 1988, 4, 274-282.
(35) Jean Chatellier, N. R.-B., Marc H. V. Van Regenmortel, Danièle Altschuh, Etienne Weiss, J. Mol. Recognit. 1996, 9, 39-51.
(36) Moudallal, Z. A.; Briand, J. P.; Regenmortel, M. H. V. V. EMBO J. 1985, 4, 1231-1235.
(37) Enander, K.; Choulier, L.; Olsson, A. L. x. a.; Yushchenko, D. A.; Kanmert, D.; Klymchenko, A. S.; Demchenko, A. P.; Mely, Y.; Altschuh, D. Bioconjugate Chem. 2008, doi: 10.1021/bc800159d.
(38) Aili, D.; Enander, K.; Baltzer, L.; and Liedberg, B Biochemical Society Transactions 2007 Volume 35, part 3, 532-534
(39) Rydberg, J. Protein-protein interactions in model systems—design, control of catalytic activity and biosensor applications. 2006 Studies in Science and Technology Dissertation no 1037, Linköping.

1. A plurality of nanoparticles, to which nanoparticles are attached
(i) first molecules that are capable of selectively binding a compound in a liquid solution; and
(ii) second molecules that have an ability to associate with one or several other second molecules attached to any other of the plurality of nanoparticles.

2. The nanoparticles according to claim 1, wherein the second molecules are selected from molecules that comprise at least one moiety enabling cation-, anion- and/or pH-induced association between said second molecules attached to different nanoparticles.

3. The nanoparticles according to claim 1, wherein the first and second molecules are polypeptides.

4. The nanoparticles according to claim 3, wherein the polypeptides are helix-loop-helix polypeptides.

5. The nanoparticles according to claim 4, wherein the helix-loop-helix polypeptides are derived from a polypeptide according to any of the SEQ. ID. NOS. 4-31 by introducing an anchoring group for attachment of the polypeptide to the nanoparticle, wherein said anchoring group is either an amino acid residue in the sequence of the helix-loop-helix polypeptides or a group attached to an amino acid residue of sequence of the helix-loop-helix polypeptides.

6. The nanoparticles according to claim 1, wherein the first and second molecules comprise a thiol, sulfide or disulfide function attached to the nanoparticle through a covalent bond.

7. The nanoparticles according to claim 3, wherein the second polypeptide molecules comprise a plurality of amino acids selected from glutamic acid and aspartic acid, such as to impart a net negative charge of from 4 to 10 to the polypeptide in a liquid solution at pH 7.

8. The nanoparticles according to claim 7, wherein the second polypeptide is derived from SEQ. ID. NO. 4.

9. The nanoparticles according to claim 8, wherein the second polypeptide is according to the SEQ. ID. NO. 2.

10. The nanoparticles according to claim 3, wherein the first polypeptide molecules have an amino acid sequence derived from any one of SEQ. ID. NOS. 4-31 by introducing an anchoring group for attachment of the polypeptide to the nanoparticle, wherein said anchoring group is either an amino acid residue of said sequence or a group attached to an amino acid residue and wherein at least one amino acid residue of the polypeptide is functionalized by attaching a moiety capable of selectively binding a compound in a liquid solution.

11. The nanoparticles according to claim 10, wherein the functionalized polypeptide comprises a lysine, ornithine, diaminobutyric acid, or homolysine residue situated in position 34 that is funtionalized.

12. The nanoparticles according to claim 10, wherein the functionalized polypeptide comprises a histidine residue in a position i and a lysine, ornithine, diaminobutyric acid, or homolysine residue situated in position i+4 and/or in position i−3 that are functionalized.

13. The nanoparticles according to claim 12, wherein i is 11.

14. The nanoparticles according to claim 10, wherein the functionalized polypeptide comprises a moiety capable of selectively binding a compound dissolved in a liquid solution that is attached to an amino acid residue through a linking chain of formula —CₙH₂ₙ—, wherein n is 1-10.

15. The nanoparticles according to claim 10, wherein the functionalized polypeptide is derived from a sequence according to any one of SEQ. ID. NOS. 23-28.

16. The nanoparticles according to claim 10, wherein the functionalized polypeptide is derived from a sequence according to any one of SEQ. ID. NOS. 29-31.

17. The nanoparticles according to claim 16, wherein the functionalized polypeptide is derived from SEQ. ID. NO. 30.

18. The nanoparticles according to claim 1, wherein the first molecules are capable of selectively binding an antibody.

19. The nanoparticles according to claim 1, wherein the first molecules are capable of selectively binding a protein, polypeptide, DNA, RNA, PNA, or carbohydrate in a liquid solution.

20. The nanoparticles according to claim 1, wherein the nanoparticles are composed of gold, silver or gold and silver alloys or a combination of gold, silver or gold and silver alloys with a material selected from other metals, semiconductors, insulators, polymers and combinations thereof.

21. The nanoparticles according to claim 1, wherein the nanoparticles are made of gold, silver or a gold/silver alloy.

22. A method for determining the presence of a compound in a liquid solution, by admixing the liquid solution with a plurality of nanoparticles according to claim 1; providing conditions effective to cause aggregation of the nanoparticles in the liquid solution in the absence of said compound in the liquid solution; and observing a detectable signal reflecting the amount of aggregation of nanoparticles in the liquid solution, wherein the presence of the compound in the liquid solution results in a detectable signal reflecting a reduced amount of aggregation of nanoparticles in the liquid solution, in comparison to the amount of aggregation of nanoparticles obtained in the liquid solution in the absence of the compound therein.

23. The method according to claim 22, wherein the conditions effective to cause aggregation of the nanoparticles in the liquid solution in the absence of said compound in the liquid solution are provided by adding a soluble salt to the liquid solution and/or by changing the pH of the liquid solution.

24. The method according to claim 23, wherein the soluble salt is a salt of a cation selected from Ca²⁺, Ni²⁺, Mg²⁺, Zn²⁺, La³⁺ and Fe³⁺.

25. The method according to claim 24, wherein the conditions effective to cause aggregation are provided by admixing the liquid solution with the plurality of nanoparticles.

26. The method according to claim 22, wherein the detectable signal is the colour of the liquid solution.

27. The method according to claim 22, wherein the detectable signal is observed in the liquid solution.

28. The method according to claim 22, comprising depositing a drop of the solution on a solid surface so as to obtain a coloured spot on said solid surface, and observing the colour of the spot on the solid surface.

29. The method according to claim 22, comprising determining the concentration of the compound in the liquid solution.

30. The method according to claim 22, wherein the compound in the liquid solution is a protein or polypeptide, DNA, RNA, PNA or carbohydrate.

31. A multi-well plate having a plurality of wells wherein each well holds a composition comprising a plurality of nanoparticles according to claim 1.

32. A kit comprising at least one container, the container holding a composition comprising a plurality of nanoparticles according to claim 1.

33. A composition comprising a plurality of nanoparticles according to claim 1 in a liquid vehicle.

34. The nanoparticles according to claim 2, wherein the first and second molecules are polypeptides.

35. The nanoparticles according to claim 11, wherein the functionalized polypeptide comprises a histidine residue in a position i and a lysine, ornithine, diaminobutyric acid, or homolysine residue situated in position i+4 and/or in position i−3 that are functionalized.


SEQUENCE LISTING (31 sequences)

SEQ ID NO: 1 - protein, 42 aa, Artificial Sequence
  Peptide KE2C
NAADLEAAIRHLAEKLAARGPCDAAQLAEQLAKKFEAFARAG

SEQ ID NO: 2 - protein, 42 aa, Artificial Sequence
  Peptide JR2EC
NAADLEKAIEALEKHLEAKGPCDAAQLEKQLEQAFEAFERAG

SEQ ID NO: 3 - protein, 19 aa, Artificial Sequence
  Peptide C-pTMVP
CRGTGSYNRSSFESSSGLV

SEQ ID NO: 4 - protein, 42 aa, Artificial Sequence
  Peptide SA-42
NXADXEAAIKALAEHXXAKGPVDXAQXAEQLAKAFEAFARXG

SEQ ID NO: 5 - protein, 42 aa, Artificial Sequence
  Peptide SA-42
NXADXEAXIXHLXEXXXERGPXDCXQXAEQLAXXFEXFARAG

SEQ ID NO: 6 - protein, 42 aa, Artificial Sequence
  Peptide SA-42
NAADLEAXIRHLAEKLAARGPXDAAQLAEQLAXXFEAFARAG

SEQ ID NO: 7 - protein, 42 aa, Artificial Sequence
  Peptide SA-42
NEADLEAKIRHLAEKLEARGPEDCEQLAEQLARAFEAFARAG

SEQ ID NO: 8 - protein, 42 aa, Artificial Sequence
  Peptide SA-42
NEADLEAKIRHLAEKLAARGPVDCAQLAEQLARAFEAFARAG

SEQ ID NO: 9 - protein, 42 aa, Artificial Sequence
  Peptide SA-42
NAADXEAKIRHLAEKXAARGPVDCAQXAEQLARRFEAFARAG

SEQ ID NO: 10 - protein, 42 aa, Artificial Sequence
  Peptide SA-42
NAADXEAKIRHLREKXAARGPRDCAQXAEQLARRFERFARAG

SEQ ID NO: 11 - protein, 42 aa, Artificial Sequence
  Peptide SA-42
NAADLEAAIKHLAEALKERGPEDCEQLAEQLARAFEAFARAG

SEQ ID NO: 12 - protein, 42 aa, Artificial Sequence
  Peptide SA-42
NAADLEAAIKHLAEALKARGPVDCAQLAEQLARAFEAFARAG

SEQ ID NO: 13 - protein, 42 aa, Artificial Sequence
  Peptide SA-42
NAADXEARIKHLAERXKARGPVDCAQXAEQLARAFEAFARAG

SEQ ID NO: 14 - protein, 42 aa, Artificial Sequence
  Peptide SA-42
NAADXEARIKHLRERXKARGPRDCAQXAEQLARAFERFARAG

SEQ ID NO: 15 - protein, 42 aa, Artificial Sequence
  Peptide SA-42
NEADLEAAIRHLAEALEARGPKDCKQLAEQLARAFEAFERAG

SEQ ID NO: 16 - protein, 42 aa, Artificial Sequence
  Peptide SA-42
NEADLEAAIRHLAEALAARGPKDCKQLAEQLARAFEAFARAG

SEQ ID NO: 17 - protein, 42 aa, Artificial Sequence
  Peptide SA-42
NAADXEAAIRHLAERXAARGPKDCKQXAEQLARAFEAFARAG

SEQ ID NO: 18 - protein, 42 aa, Artificial Sequence
  Peptide SA-42
NAADXEARIRHLRERXAARGPKDCKQXAEQLARAFERFARAG

SEQ ID NO: 19 - protein, 42 aa, Artificial Sequence
  Peptide SA-42
NEADLEAAIRHLAERLEARGPADCAQLAEQLAAKFEKFARAG

SEQ ID NO: 20 - protein, 42 aa, Artificial Sequence
  Peptide SA-42
NAADLEAAIRHLAERLAARGPVDCAQLAEQLAAKFEKFARAG

SEQ ID NO: 21 - protein, 42 aa, Artificial Sequence
  Peptide SA-42
NAADXEAAIRHLAERXAARGPVDCAQXAEQLARKFEKFARAG

SEQ ID NO: 22 - protein, 42 aa, Artificial Sequence
  Peptide SA-42
NAADXEAKIRHLRERXAARGPRECAQXAEQLARKFEKFARAG

SEQ ID NO: 23 - protein, 42 aa, Artificial Sequence
  Peptide SA-42
NAADXEAAIKALAEAXAAKGPVDAAQXAEQLAKXFEAFARAG

SEQ ID NO: 24 - protein, 42 aa, Artificial Sequence
  Peptide KA-I-A33
NAADXEAAIKALAEKXAAKGPVDAAQXAEQLAAXFEAFARAG

SEQ ID NO: 25 - protein, 42 aa, Artificial Sequence
  Peptide KA-I-R19
NAADXEAAIKALAEKXAARGPVDAAQXAEQLAKXFEAFARAG

SEQ ID NO: 26 - protein, 42 aa, Artificial Sequence
  Peptide KA-II
NAADXEAAIKAKLEKXAARGPVDAAQXAEQLAKAFEAFARAG

SEQ ID NO: 27 - protein, 42 aa, Artificial Sequence
  Peptide LA-42b
NAADXEAAIKHLAEKXAAKGPVDAAQXAEQLAKXFEAFARAG

SEQ ID NO: 28 - protein, 42 aa, Artificial Sequence
  Peptide LA-42h
NAADXEAKIRHLAEKXAARGPVDAAQXAEQLAKKFEAFARAG

SEQ ID NO: 29 - protein, 42 aa, Artificial Sequence; identical to SEQ ID NO: 6
  Peptide KE2C

SEQ ID NO: 30 - protein, 42 aa, Artificial Sequence
  Peptide KE2
NAADLEAAIRHLAEKLAARGPVDAAQLAEQLAKKFEAFARAG

SEQ ID NO: 31 - protein, 42 aa, Artificial Sequence
  Peptide KE3
NAADLEAKIRHLAEKLAARGPCDAAQLAEQLARRFEAFARAG